(19) United States
(12) Patent Application Publication
(10) Pub. No.: US 2021/0138042 A1
(43) Pub. Date: May 13, 2021

(54) FORMULATIONS FOR NUTRITIONAL SUPPORT IN SUBJECTS IN NEED THEREOF

(71) Applicant: ByHeart, Inc., New York, NY (US)

(72) Inventors: Ron Belldegrun, New York, NY (US); Mia Funt, New York, NY (US)

(73) Assignee: ByHeart, Inc., New York, NY (US)

(21) Appl. No.: 17/155,759

(22) Filed: Jan. 22, 2021

Related U.S. Application Data
(63) Continuation of application No. 16/428,589, filed on May 31, 2019, now abandoned.
Application No. 16/428,589 is a continuation-in-part of application No. PCT/US2018/063427, filed on Nov. 30, 2018.
Application No. PCT/US2018/063427 is a continuation-in-part of application No. 15/829,703, filed on Dec. 1, 2017.

(51) Int. Cl. A61K 38/40; A61K 38/19; A61K 38/38; A23L 33/19; A61K 38/01; A23L 33/00; A61P 1/14

(52) U.S. Cl. CPC A61K 38/40; A61K 38/19; A61K 38/38; A61P 1/14; A61K 38/018; A23L 33/40; A23L 33/19

(57) ABSTRACT

Formulations having a protein component, in which the protein contains one or more digestion-aiding proteins, and one or more immunoprotective proteins. The ratio by weight of the one or digestion-aiding proteins to the one or more immunoprotective proteins may be about 12:1 to about 1:1. The formulations may also contain a fat component, a carbohydrate component, and vitamins and minerals. These formulations can be used to provide nutritional support to a subject, either as dietary supplements or as a primary source of nutrition, such as for an infant formula. The formulations may also be used to promote or induce proliferation of intestinal cells, promote or induce differentiation of intestinal cells, prevent or inhibit growth of enteropathogenic Escherichia coli in the digestive system of a subject, prevent or inhibit bacterial growth in the intestinal lumen, increase interleukin-18 secretion by intestinal cells, or increase intestinal immunity.

### CROSS-REFERENCE TO RELATED APPLICATIONS

This application is a continuation-in-part of International Application No. PCT/US2018/063427, filed on Nov. 30, 2018, and is a continuation-in-part of U.S. patent application Ser. No. 15/829,703, filed on Dec. 1, 2017. Both applications are hereby incorporated by reference in their entirety.

### FIELD OF THE DISCLOSURE

The disclosure relates to formulations that can provide nutritional support to a subject, for example, as a dietary supplement or as a primary source of nutrition. In some applications, the formulation may be used in an exempt infant formula, non-exempt infant formula, or medical food.

### BACKGROUND

While it is generally recommended that infants are fed breast milk, in many cases breast feeding is not feasible or adequate, or the parent(s) choose not to breast feed. To this end, infant formulas have been developed with the primary objective of providing the nutrition that infants would otherwise receive from breast milk.

However, many infant formulas on the market are not formulated to provide the full nutritional and health benefits seen with breast milk, which is primarily due to the proteins included in these formulas. For example, research in this area has shown that certain intact proteins—and their peptides that are naturally produced through the digestive process—provide bioactivity that may be important for the development of a baby's immune system, and support for anti-viral, antibacterial, immunomodulatory and anti-inflammatory effects. These proteins are often absent or in insufficient quantities in current infant formulas. Moreover, cow-based infant formulas are often missing proteases that help break down proteins and aid in digestion, which can make those infant formulas difficult to digest and tolerate.

Some infant formulas on the market today are formulated to address either digestion comfort or immunity, but not both. The products formulated for digestive comfort are typically 100% partially hydrolyzed from whey protein, which may improve tolerability. But these formulas are made up of nearly all protein hydrolysates, and may neglect important bioactive peptides that are a bi-product of digestion of intact protein. These 100% hydrolyzed formulas also lack important intact bioactive proteins present in both bovine milk and human milk, such as lactoferrin and osteopontin, which have important immunity-protective properties. On the other hand, the few infant formulas that incorporate bioactive protein supporting immunity are typically made up of only intact proteins and are not specifically formulated for easy digestion, such as by including partially hydrolyzed proteins. In addition, many current infant formulas do not limit the bovine-based proteins, such as beta-lactaglobulin, which are not in human milk and which may be a source of the digestive sensitivity to the formula.

Thus, there is a continuing need to develop infant formulas that are easy to digest and that provide the nutritional and bioactive benefits of breast milk.

### SUMMARY OF INVENTION

One aspect of the invention relates to a formulation for oral administration comprising a protein component, in which the protein component comprises whey protein hydrolysate (WPH) protein and alpha-lactalbumin.

In some embodiments, the WPH protein and the alpha-lactalbumin are present in a ratio of about 4:1 to about 1:1, or about 7:2 to about 5:4, by weight.

In certain embodiments, the WPH protein and the alpha-lactalbumin comprise about 15% to about 95%, or about 30% to about 70%, or about 60% to about 90%, or about 60% to about 75%, or about 75% to about 90%, by weight of the protein component. In certain embodiments, the WPH protein and the alpha-lactalbumin comprise about 40%, or about 44%, or about 49%, or about 53%, or about 59%, or about 63%, or about 67%, or about 70%, or about 76%, or about 83%, or about 88%, by weight of the protein component.

In some embodiments, the WPH protein comprises about 10% to about 75%, or about 20% to about 50%, or about 45% to about 70%, or about 45% to about 60%, or about 55% to about 70%, by weight of the protein component. In certain embodiments, the WPH protein comprises about 26%, or about 35%, or about 45%, or about 49%, or about 52%, or about 55%, or about 59%, or about 65%, or about 69%, by weight of the protein component.

In some embodiments, the alpha-lactalbumin comprises about 5% to about 25%, or about 10% to about 20%, or about 12% to about 20%, or about 13% to about 16%, or about 15% to about 20%, by weight of the protein component. In certain embodiments, the alpha-lactalbumin comprises about 14%, or about 15%, or about 17%, or about 18%, or about 19%, by weight of the protein component.

In certain embodiments, the WPH protein comprises about 26% or about 35% or about 45% by weight of the protein component, and the alpha-lactalbumin comprises about 14% or about 18% by weight of the protein component.

The protein component may further comprise one or more immunoprotective proteins. In some embodiments, the one or more immunoprotective proteins are selected from the group consisting of lactoferrin, osteopontin, x-casein, haptocorrin, lysozyme, secretory IgA, and bile-salt stimulated lipase. In some embodiments, the one or more immunoprotective proteins comprise lactoferrin, osteopontin, or both lactoferrin and osteopontin.

In some embodiments, the lactoferrin comprises about 4% to about 30%, or about 6% to about 7%, or about 5% to about 25%, or about 6% to about 20%, or about 8% to about 22%, by weight of the protein component. In certain embodiments, the lactoferrin comprises about 6%, or about 7%, or about 10%, or about 12%, or about 15%, or about 18%, or about 22%, by weight of the protein component.

In some embodiments, the osteopontin comprises about 0.1% to about 4%, or about 0.5% to about 1.5%, or about 1% to about 3%, or about 1.5% to about 2%, or about 2% to about 2.5%, by weight of the protein component. In certain embodiments, the osteopontin comprises about 1%, or about 1.5%, or about 2%, or about 2.5%, by weight of the protein component.

In some embodiments, the protein component comprises about 5% to about 20%, or about 8% to about 16%, by weight of the formulation. In certain embodiments, the protein component comprises about 10% or about 15% by weight of the formulation.

The formulations may further comprise a fat component, a milk component, a carbohydrate component, or a combination thereof.

In some embodiments, the fat component comprises innate milk fat globule membrane (MFGM), added MFGM, phospholipids, cholesterol, oil, non-hexane extracted docosahexaenoic acid (DHA), hexane extracted arachidonic acid (AA), non-hexane extracted AA, or a combination thereof. In certain embodiments, the oil comprises vegetable oil, soy oil, palm oil, or a combination thereof.

In some embodiments, the milk component comprises milk from a non-human source, such as bovine milk. In certain embodiments, the milk comprises whole milk. In some embodiments, the milk is in an amount to provide about 1% to about 40%, or about 8% to about 35%, or about 12% to about 30%, by weight of the protein component. In certain embodiments, the milk is in an amount to provide about 16%, or about 18%, or about 25%, by weight of the protein component.

In some embodiments, the carbohydrate component comprises lactose, galactooligosaccharide (GOS), fructooligosaccharide (FOS), inulin, corn syrup solids, maltodextrin, or a combination thereof.

In some embodiments, the formulation is in a powder form.

Another aspect of the present invention relates to a formulation for oral administration comprising a protein component that comprises lactoferrin and osteopontin, in which the lactoferrin and osteopontin comprise about 4% to about 35% by weight of the protein component, and the formulation further comprises a milk component comprising milk from a non-human source.

In some embodiments, the lactoferrin and osteopontin are present in a ratio of about 12:1 to about 2:1, or about 9:1 to about 6:1, or about 11:1 to about 3:1, by weight. In some embodiments, the lactoferrin and osteopontin are present in a molecular ratio of about 10:1 to about 2:1, or about 8:1 to about 3:1, such as about 8:1, or about 5:1, or about 3:1.

In some embodiments, the lactoferrin and osteopontin comprise about 4% to about 35%, or about 6% to about 9%, or about 5% to about 30%, or about 8% to about 20%, or about 10% to about 25%, by weight of the protein component. In certain embodiments, the lactoferrin and osteopontin comprise about 7% to about 8%, or about 10%, or about 12%, or about 14%, or about 17%, or about 20%, or about 24%, by weight of the protein component.

In some embodiments, the lactoferrin comprises about 4% to about 30%, or about 6% to about 7%, or about 5% to about 25%, or about 6% to about 20%, or about 8% to about 22%, by weight of the protein component. In certain embodiments, the lactoferrin comprises about 6%, or about 7%, or about 10%, or about 12%, or about 15%, or about 18%, or about 22%, by weight of the protein component.

In some embodiments, the osteopontin comprises about 0.1% to about 4%, or about 0.5% to about 1.5%, or about 1% to about 3%, or about 1.5% to about 2%, or about 2% to about 2.5%, by weight of the protein component. In certain embodiments, the osteopontin comprises about 1%, or about 1.5%, or about 2%, or about 2.5%, by weight of the protein component.

In certain embodiments, the lactoferrin comprises about 6% or about 7% by weight of the protein component, and the osteopontin comprises about 1% by weight of the protein component.

In embodiments of the invention, the protein component further comprises one or more digestion-aiding proteins. In certain embodiments, the one or more digestion-aiding proteins comprise WPH protein, alpha-lactalbumin, or both WPH protein and alpha-lactalbumin.

In some embodiments, the WPH protein comprises about 10% to about 75%, or about 20% to about 50%, or about 45% to about 70%, or about 45% to about 60%, or about 55% to about 70%, by weight of the protein component. In certain embodiments, the WPH protein comprises about 26%, or about 35%, or about 45%, or about 49%, or about 52%, or about 55%, or about 59%, or about 65%, or about 69%, by weight of the protein component.

In some embodiments, the alpha-lactalbumin comprises about 5% to about 25%, or about 10% to about 20%, or about 12% to about 20%, or about 13% to about 16%, or about 15% to about 20%, by weight of the protein component. In certain embodiments, the alpha-lactalbumin comprises about 14%, or about 15%, or about 17%, or about 18%, or about 19%, by weight of the protein component.

In some embodiments, the protein component comprises about 5% to about 20%, or about 8% to about 16%, by weight of the formulation. In certain embodiments, the protein component comprises about 10% or about 15% by weight of the formulation.

The formulations may further comprise a fat component, a milk component, a carbohydrate component, or a combination thereof.

In some embodiments, the fat component comprises innate MFGM, added MFGM, phospholipids, cholesterol, oil, non-hexane extracted DHA, hexane extracted AA, non-hexane extracted AA, or a combination thereof. In certain embodiments, the oil comprises vegetable oil, soy oil, palm oil, or a combination thereof.

In some embodiments, the milk component comprises milk from a non-human source, such as bovine milk. In certain embodiments, the milk comprises whole milk. In some embodiments, the milk is in an amount to provide about 1% to about 40%, or about 8% to about 35%, or about 12% to about 30%, by weight of the protein component. In certain embodiments, the milk is in an amount to provide about 16%, or about 18%, or about 25%, by weight of the protein component.

In some embodiments, the carbohydrate component comprises lactose, GOS, FOS, inulin, corn syrup solids, maltodextrin, or a combination thereof.

In some embodiments, the formulation is in a powder form.

An aspect of the present invention relates to a formulation for oral administration comprising a protein component, in which the protein component comprises: (a) one or more digestion-aiding proteins, selected from the group consisting of WPH protein, alpha-lactalbumin, and K-casein; and (b) one or more immunoprotective proteins, selected from the group consisting of lactoferrin, osteopontin, K-casein, haptocorrin, lysozyme, secretory IgA, and bile-salt stimulated lipase; in which the one or more digestion-aiding proteins and the one or more immunoprotective proteins comprise about 5% to about 95% by weight of the protein component; and the formulation further comprises a milk component comprising milk from a non-human source.

In some embodiments, the one or more digestion-aiding proteins and the one or more immunoprotective proteins in the formulation are present in a ratio of about 12:1 to about 1:1, or about 10:1 to about 3:1, or about 8:1 to about 3:1, or about 10:1 to about 4:1, by weight.

In some embodiments, the one or more digestion-aiding proteins comprise WPH protein, and the one or more immunoprotective proteins comprise lactoferrin. In certain embodiments, the WPH protein and lactoferrin are present in a ratio of about 9:1 to about 1:1, or about 8:1 to about 2:1, or about 7:1 to about 5:2, by weight.

In some embodiments, the WPH protein comprises about 10% to about 75%, or about 20% to about 50%, or about 45% to about 70%, or about 45% to about 60%, or about 55% to about 70%, by weight of the protein component. In certain embodiments, the WPH protein comprises about 26%, or about 35%, or about 45%, or about 49%, or about 52%, or about 55%, or about 59%, or about 65%, or about 69%, by weight of the protein component.

In some embodiments, the lactoferrin comprises about 4% to about 30%, or about 6% to about 7%, or about 5% to about 25%, or about 6% to about 20%, or about 8% to about 22%, by weight of the protein component. In certain embodiments, the lactoferrin comprises about 6%, or about 7%, or about 10%, or about 12%, or about 15%, or about 18%, or about 22%, by weight of the protein component.

In some embodiments, the one or more digestion-aiding proteins comprise WPH protein, and the one or more immunoprotective proteins comprise osteopontin. In certain embodiments, the WPH protein and osteopontin are present in a ratio of about 60:1 to about 10:1, or about 50:1 to about 25:1, by weight.

In some embodiments, the WPH protein comprises about 10% to about 75%, or about 20% to about 50%, or about 45% to about 70%, or about 45% to about 60%, or about 55% to about 70%, by weight of the protein component. In certain embodiments, the WPH protein comprises about 26%, or about 35%, or about 45%, or about 49%, or about 52%, or about 55%, or about 59%, or about 65%, or about 69%, by weight of the protein component.

In some embodiments, the osteopontin comprises about 0.1% to about 4%, or about 0.5% to about 1.5%, or about 1% to about 3%, or about 1.5% to about 2%, or about 2% to about 2.5%, by weight of the protein component. In certain embodiments, the osteopontin comprises about 1%, or about 1.5%, or about 2%, or about 2.5%, by weight of the protein component.

In some embodiments, the one or more digestion-aiding proteins comprise alpha-lactalbumin, and the one or more immunoprotective proteins comprise lactoferrin. In certain embodiments, the alpha-lactalbumin and lactoferrin are present in a ratio of about 4:1 to about 0.5:1, or about 3:1 to about 2:1, or about 2:1 to about 0.8:1, by weight.

In some embodiments, the alpha-lactalbumin comprises about 5% to about 25%, or about 10% to about 20%, or about 12% to about 20%, or about 13% to about 16%, or about 15% to about 20%, by weight of the protein component. In certain embodiments, the alpha-lactalbumin comprises about 14%, or about 15%, or about 17%, or about 18%, or about 19%, by weight of the protein component.

In some embodiments, the lactoferrin comprises about 4% to about 30%, or about 6% to about 7%, or about 5% to about 25%, or about 6% to about 20%, or about 8% to about 22%, by weight of the protein component. In certain embodiments, the lactoferrin comprises about 6%, or about 7%, or about 10%, or about 12%, or about 15%, or about 18%, or about 22%, by weight of the protein component.

In some embodiments, the one or more digestion-aiding proteins comprise alpha-lactalbumin, and the one or more immunoprotective proteins comprise osteopontin. In certain embodiments, the alpha-lactalbumin and osteopontin are present in a ratio of about 25:1 to about 5:1, or about 20:1 to about 7:1, by weight of the protein component.

In some embodiments, the alpha-lactalbumin comprises about 5% to about 25%, or about 10% to about 20%, or about 12% to about 20%, or about 13% to about 16%, or about 15% to about 20%, by weight of the protein component. In certain embodiments, the alpha-lactalbumin comprises about 14%, or about 15%, or about 17%, or about 18%, or about 19%, by weight of the protein component.

In some embodiments, the osteopontin comprises about 0.1% to about 4%, or about 0.5% to about 1.5%, or about 1% to about 3%, or about 1.5% to about 2%, or about 2% to about 2.5%, by weight of the protein component. In certain embodiments, the osteopontin comprises about 1%, or about 1.5%, or about 2%, or about 2.5%, by weight of the protein component.

In some embodiments, the protein component comprises about 5% to about 20%, or about 8% to about 16%, by weight of the formulation. In certain embodiments, the protein component comprises about 10% or about 15% by weight of the formulation.

The formulations may further comprise a fat component, a milk component, a carbohydrate component, or a combination thereof.

In some embodiments, the fat component comprises innate MFGM, added MFGM, phospholipids, cholesterol, oil, non-hexane extracted DHA, hexane extracted AA, non-hexane extracted AA, or a combination thereof. In certain embodiments, the oil comprises vegetable oil, soy oil, palm oil, or a combination thereof.

In some embodiments, the milk component comprises milk from a non-human source, such as bovine milk. In certain embodiments, the milk comprises whole milk. In some embodiments, the milk is in an amount to provide about 1% to about 40%, or about 8% to about 35%, or about 12% to about 30%, by weight of the protein component. In certain embodiments, the milk is in an amount to provide about 16%, or about 18%, or about 25%, by weight of the protein component.

In some embodiments, the carbohydrate component comprises lactose, GOS, FOS, inulin, corn syrup solids, maltodextrin, or a combination thereof.

In some embodiments, the formulation is in a powder form.

A further aspect of the present invention relates to a formulation for oral administration comprising a protein component and a fat component, in which the protein component comprises osteopontin, and the fat component comprises MFGM, and in which the formulation comprises a milk component comprising milk from a non-human source.

In some embodiments, the osteopontin comprises about 0.1% to about 4%, or about 0.5% to about 1.5%, or about 1% to about 3%, or about 1.5% to about 2%, or about 2% to about 2.5%, by weight of the protein component. In certain embodiments, the osteopontin comprises about 1%, or about 1.5%, or about 2%, or about 2.5%, by weight of the protein component.

In some embodiments, the protein component further comprises lactoferrin.

In some embodiments, the lactoferrin comprises about 4% to about 30%, or about 6% to about 7%, or about 5% to about 25%, or about 6% to about 20%, or about 8% to about 22%, by weight of the protein component. In certain embodiments, the lactoferrin comprises about 6%, or about 7%, or about 10%, or about 12%, or about 15%, or about 18%, or about 22%, by weight of the protein component.

In certain embodiments, the protein component further comprises one or more digestion-aiding proteins, such as WPH protein, alpha-lactalbumin, or both WPH protein and alpha-lactalbumin.

In some embodiments, the WPH protein comprises about 10% to about 75%, or about 20% to about 50%, or about 45% to about 70%, or about 45% to about 60%, or about 55% to about 70%, by weight of the protein component. In certain embodiments, the WPH protein comprises about 26%, or about 35%, or about 45%, or about 49%, or about 52%, or about 55%, or about 59%, or about 65%, or about 69%, by weight of the protein component.

In some embodiments, the alpha-lactalbumin comprises about 5% to about 25%, or about 10% to about 20%, or about 12% to about 20%, or about 13% to about 16%, or about 15% to about 20%, by weight of the protein component. In certain embodiments, the alpha-lactalbumin comprises about 14%, or about 15%, or about 17%, or about 18%, or about 19%, by weight of the protein component.

In some embodiments, the MFGM is innate or added.

In some embodiments, the fat component further comprises phospholipids, cholesterol, oil, non-hexane extracted DHA, hexane extracted AA, non-hexane extracted AA, or a combination thereof. In certain embodiments, the oil comprises vegetable oil, soy oil, palm oil, or a combination thereof.

In some embodiments, the milk comprises bovine milk. In certain embodiments, the milk comprises whole milk.

In some embodiments, the milk is in an amount to provide about 1% to about 40%, or about 8% to about 35%, or about 12% to about 30%, by weight of the protein component. In certain embodiments, the milk is in an amount to provide about 16%, or about 18%, or about 25%, by weight of the protein component.

In some embodiments, the formulation comprises a carbohydrate component. In certain embodiments, the carbohydrate component comprises lactose, GOS, FOS, inulin, corn syrup solids, maltodextrin, or a combination thereof.

In some embodiments, the formulation is in powder form.

An aspect of the invention relates to a formulation for oral administration comprising a protein component, a fat component, and a carbohydrate component, in which (a) the protein component comprises WPH protein, alpha-lactalbumin, lactoferrin, and osteopontin; (b) the fat component comprises MFGM, phospholipids, cholesterol, non-hexane extracted DHA, and either hexane extracted AA or non-hexane extracted AA; and (c) the carbohydrate component comprises lactose and GOS.

An aspect of the invention relates to a formulation for oral administration comprising a protein component, a fat component, and a carbohydrate component, in which (a) the protein component comprises WPH protein, alpha-lactalbumin, and lactoferrin; (b) the fat component comprises MFGM, phospholipids, cholesterol, non-hexane extracted DHA, and either hexane extracted AA or non-hexane extracted AA; and (c) the carbohydrate component comprises lactose and GOS.

A further aspect of the invention relates to a formulation for oral administration comprising a protein component, a fat component, a carbohydrate component, and milk, in which (a) the protein component comprises WPH protein, alpha-lactalbumin, lactoferrin, and osteopontin, such that the WPH protein comprises about 35% by weight of the protein component, the alpha-lactalbumin comprises about 18% by weight of the protein component, the lactoferrin comprises about 6% by weight of the protein component, and the osteopontin comprises about 1% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 16% by weight of the protein component.

Yet another aspect of the invention relates to a formulation for oral administration comprising a protein component, a fat component, a carbohydrate component, and milk, in which (a) the protein component comprises WPH protein, alpha-lactalbumin, lactoferrin, and osteopontin, such that the WPH protein comprises about 45% by weight of the protein component, the alpha-lactalbumin comprises about 18% by weight of the protein component, the lactoferrin comprises about 6% by weight of the protein component, and the osteopontin comprises about 1% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 18% by weight of the protein component.

Another aspect of the invention relates to a formulation for oral administration comprising a protein component, a fat component, a carbohydrate component, and milk, in which (a) the protein component comprises WPH protein, alpha-lactalbumin, lactoferrin, and osteopontin, such that the WPH protein comprises about 26% by weight of the protein component, the alpha-lactalbumin comprises about 18% by weight of the protein component, the lactoferrin comprises about 6% by weight of the protein component, and the osteopontin comprises about 1% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 25% by weight of the protein component.

Moreover, an aspect of the invention relates to a formulation for oral administration comprising a protein component, a fat component, a carbohydrate component, and milk, in which (a) the protein component comprises WPH protein, alpha-lactalbumin, lactoferrin, and osteopontin, such that the WPH protein comprises about 45% by weight of the protein component, the alpha-lactalbumin comprises about 14% by weight of the protein component, the lactoferrin comprises about 7% by weight of the protein component, and the osteopontin comprises about 1% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 16% by weight of the protein component.

Another aspect of the invention relates to a formulation for oral administration comprising a protein component, a fat component, a carbohydrate component, and milk, in which (a) the protein component comprises WPH protein, alpha-lactalbumin, lactoferrin, and osteopontin, such that the WPH protein comprises about 45% to about 70% by weight of the protein component, the alpha-lactalbumin comprises about 10% to about 20% by weight of the protein component, the lactoferrin comprises about 5% to about 25% by weight of the protein component, and the osteopontin comprises about 1% to about 3% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 1% to about 25% by weight of the protein component.

Yet another aspect of the invention relates to a formulation for oral administration comprising a protein component, a fat component, a carbohydrate component, and milk, in which (a) the protein component comprises WPH protein, alpha-lactalbumin, and lactoferrin, such that the WPH protein comprises about 45% to about 70% by weight of the protein component, the alpha-lactalbumin comprises about 10% to about 20% by weight of the protein component, and the lactoferrin comprises about 5% to about 25% by weight of the protein component; (b) the fat component comprises MFGM, phospholipids, cholesterol, non-hexane extracted DHA, and either hexane extracted AA or non-hexane extracted AA; (c) the carbohydrate component comprises lactose and GOS; and (d) the whole milk is in an amount to provide about 1% to about 25% by weight of the protein component.

A further aspect of the present invention relates to methods involving the formulations described herein. In some embodiments, the present invention relates to a method of providing nutritional support to a subject in need thereof, the method comprising administering to the subject a formulation of the present invention.

In some embodiments, the present invention relates to a method of promoting proliferation of intestinal cells in a subject in need thereof, the method comprising administering to the subject a formulation of the present invention.

In some embodiments, the present invention relates to a method of inducing proliferation of intestinal cells in a subject in need thereof, the method comprising administering to the subject a formulation of the present invention.

In some embodiments, the present invention relates to a method of promoting differentiation of intestinal cells in a subject in need thereof, the method comprising administering to the subject a formulation of the present invention.

In some embodiments, the present invention relates to a method of inducing differentiation of intestinal cells in a subject in need thereof, the method comprising administering to the subject a formulation of the present invention.

In some embodiments, the present invention relates to a method of preventing growth of enteropathogenic Escherichia coli (EPEC) in the digestive system of a subject in need thereof, the method comprising administering to the subject a formulation of the present invention.

In some embodiments, the present invention relates to a method of inhibiting growth of EPEC in the digestive system of a subject in need thereof, the method comprising administering to the subject a formulation of the present invention.

In some embodiments, the present invention relates to a method of preventing bacterial infection in the intestinal lumen of a subject in need thereof, the method comprising administering to the subject the formulation of the present invention. In certain embodiments, the bacterial infection is prevented by preventing or inhibiting growth of bacteria.

In some embodiments, the present invention relates to a method of inhibiting bacterial infection in the intestinal lumen of a subject in need thereof, the method comprising administering to the subject the formulation of the present invention. In certain embodiments, the bacterial infection is inhibited by inhibiting growth of bacteria.

In some embodiments, the present invention relates to a method of increasing interleukin-18 (IL-18) secretion by intestinal cells in a subject in need thereof, the method comprising administering to the subject a formulation of the present invention.

In some embodiments, the present invention relates to a method of increasing intestinal immunity in a subject in need thereof, the method comprising administering to the subject a formulation of the present invention.

An aspect of the present invention relates to uses of the formulations described herein. In some embodiments, the present invention relates to use of a formulation of the present invention to provide nutritional support to a subject in need thereof, in which the use comprises administering to the subject the formulation of the present invention.

In some embodiments, the present invention relates to use of a formulation of the present invention to promote proliferation of intestinal cells in a subject in need thereof, in which the use comprises administering to the subject the formulation of the present invention.

In some embodiments, the present invention relates to use of a formulation of the present invention to induce proliferation of intestinal cells in a subject in need thereof, in which the use comprises administering to the subject the formulation of the present invention.

In some embodiments, the present invention relates to use of a formulation of the present invention to promote differentiation of intestinal cells in a subject in need thereof, in which the use comprises administering to the subject the formulation of the present invention.

In some embodiments, the present invention relates to use of a formulation of the present invention to induce differentiation of intestinal cells in a subject in need thereof, in which the use comprises administering to the subject the formulation of the present invention.

In some embodiments, the present invention relates to use of a formulation of the present invention to prevent growth of EPEC in the digestive system of a subject in need thereof, in which the use comprises administering to the subject the formulation of the present invention.

In some embodiments, the present invention relates to use of a formulation of the present invention to inhibit growth of EPEC in the digestive system of a subject in need thereof, in which the use comprises administering to the subject the formulation of the present invention.

In some embodiments, the present invention relates to use of a formulation of the present invention to prevent bacterial infection in the intestinal lumen of a subject in need thereof, in which the use comprises administering to the subject the formulation of the present invention. In certain embodiments, the bacterial infection is prevented by preventing or inhibiting growth of bacteria.

In some embodiments, the present invention relates to use of a formulation of the present invention to inhibit bacterial infection in the intestinal lumen of a subject in need thereof, in which the use comprises administering to the subject the formulation of the present invention. In certain embodiments, the bacterial infection is inhibited by inhibiting growth of bacteria.

In some embodiments, the present invention relates to use of a formulation of the present invention to increase IL-18 secretion by intestinal cells in a subject in need thereof, in which the use comprises administering to the subject the formulation of the present invention.

In some embodiments, the present invention relates to use of a formulation of the present invention to increase intestinal immunity in a subject in need thereof, in which the use comprises administering to the subject the formulation of the present invention.

An aspect of the present invention relates to a formulation of the present invention for various uses. In some embodiments, the present invention relates to a formulation of the present invention for use in providing nutritional support to a subject in need thereof, in which the formulation is administered to the subject.

In some embodiments, the present invention relates to a formulation of the present invention for use in promoting proliferation of intestinal cells in a subject in need thereof, in which the use comprises administering to the subject the formulation of the present invention.

In some embodiments, the present invention relates to a formulation of the present invention for use in inducing proliferation of intestinal cells in a subject in need thereof, in which the use comprises administering to the subject the formulation of the present invention.

In some embodiments, the present invention relates to a formulation of the present invention for use in promoting differentiation of intestinal cells in a subject in need thereof, in which the use comprises administering to the subject the formulation of the present invention.

In some embodiments, the present invention relates to a formulation of the present invention for use in inducing differentiation of intestinal cells in a subject in need thereof, in which the use comprises administering to the subject the formulation of the present invention.

In some embodiments, the present invention relates to a formulation of the present invention for use in preventing growth of EPEC in the digestive system of a subject in need thereof, in which the use comprises administering to the subject the formulation of the present invention.

In some embodiments, the present invention relates to a formulation of the present invention for use in inhibiting growth of EPEC in the digestive system of a subject in need thereof, in which the use comprises administering to the subject the formulation of the present invention.

In some embodiments, the present invention relates to a formulation of the present invention for use in preventing bacterial infection in the intestinal lumen of a subject in need thereof, in which the use comprises administering to the subject the formulation of the present invention. In certain embodiments, the bacterial infection is prevented by preventing or inhibiting growth of bacteria.

In some embodiments, the present invention relates to a formulation of the present invention for use in inhibiting bacterial infection in the intestinal lumen of a subject in need thereof, in which the use comprises administering to the subject the formulation of the present invention. In certain embodiments, the bacterial infection is inhibited by preventing or inhibiting growth of bacteria.

In some embodiments, the present invention relates to a formulation of the present invention for use in increasing IL-18 secretion by intestinal cells in a subject in need thereof, in which the use comprises administering to the subject the formulation of the present invention.

In some embodiments, the present invention relates to a formulation of the present invention for use in increasing intestinal immunity in a subject in need thereof, in which the use comprises administering to the subject the formulation of the present invention.

### BRIEF DESCRIPTION OF THE DRAWING FIGURES

Reference is made to the following description of exemplary embodiments of the present invention, and to the accompanying drawings, wherein:

FIG. 1 shows the results of using Blue Native PAGE to identify the lactoferrin-osteopontin complex in formulations studied in Example 3 (LF=lactoferrin; OPN=osteopontin).

FIG. 2 shows the results of using Native PAGE and immunoblotting with a lactoferrin antibody to identify the lactoferrin-osteopontin complex in formulations studied in Example 3 (LF=lactoferrin; OPN=osteopontin).

FIG. 3 shows the results of using Native PAGE and immunoblotting with an osteopontin antibody to identify the lactoferrin-osteopontin complex in formulations studied in Example 3 (LF=lactoferrin; OPN=osteopontin).

FIG. 4 shows the results of using SDS PAGE and Western blotting with a lactoferrin antibody to identify the lactoferrin-osteopontin complex in formulations studied in Example 3 (LF=lactoferrin; OPN=osteopontin).

FIG. 5 shows the results of using SDS PAGE and Western blotting with an osteopontin antibody to identify the lactoferrin-osteopontin complex in formulations studied in Example 3 (LF=lactoferrin; OPN=osteopontin).

FIG. 6 shows the results of using SDS PAGE and Western blotting with a lactoferrin antibody to evaluate in vitro digestion in formulations studied in Example 3 (LF=lactoferrin).

FIG. 7 shows the results of using SDS PAGE and Western blotting with an osteopontin antibody to evaluate in vitro digestion in formulations studied in Example 3 (OPN=osteopontin).

FIG. 8 shows the results from an evaluation on cell proliferation of human intestinal crypt-like cells (HIECs) following treatment with formulations studied in Example 3 (LF=lactoferrin; OPN=osteopontin).

FIG. 9 shows the results from an evaluation on cell differentiation of human colon adenocarcinoma (Caco-2) cells following treatment with formulations studied in Example 3 (LF=lactoferrin; OPN=osteopontin).

FIG. 10 shows the results from an evaluation on lactoferrin binding of Caco-2 cells following treatment with formulations studied in Example 3 (LF=lactoferrin; OPN=osteopontin).

FIG. 11 shows the results from an evaluation on lactoferrin uptake of Caco-2 cells following treatment with formulations studied in Example 3 (LF=lactoferrin; OPN=osteopontin).

FIGS. 12A-12C show the results from an evaluation on growth of enteropathogenic Escherichia coli following treatment with formulations studied in Example 3 (LF=lactoferrin; OPN=osteopontin). FIG. 12A shows the results from treatment with lactoferrin-only formulations at the quantities for the 3:1, 5:1, and 8:1 ratios; FIG. 12B shows the results from treatment with lactoferrin+osteopontin mixtures at the 3:1, 5:1, and 8:1 ratios; and FIG. 12C shows the results from treatment with protein blends containing lactoferrin and osteopontin at the 3:1, 5:1, and 8:1 ratios.

FIG. 13 shows the results from an evaluation on secretion of interleukin-18 by Caco-2 cells following treatment with formulations studied in Example 3 (LF=lactoferrin; OPN=osteopontin).

### DETAILED DESCRIPTION

The present invention relates to formulations comprising a protein component, in which the protein component comprises one or more digestion-aiding proteins, and/or one or more immunoprotective proteins. These formulations can be used to provide nutritional support to a subject, either as dietary supplements or as a primary source of nutrition. For example, the formulations may be used as a nutritional supplement, children's nutritional product, infant formula, human milk fortifier, growing-up milk, or any other nutritional composition designed for an infant or a pediatric subject. In particular embodiments, these formulations may be used as an infant formula.

### Combination of Digestion-Aiding Proteins

One aspect of the invention relates to a formulation comprising a protein component, in which the protein component comprises two or more digestion-aiding proteins. A digestion-aiding protein is a protein that is easy to digest and is tolerated by the body. Formulations that are easy to digest are especially important for infants who may be relying on formulas as their primary or sole source of nutrition.

The protein component of a formulation of the present invention may comprise partially hydrolyzed proteins. The hydrolyzed proteins may be treated with enzymes to break down some or most of the proteins that cause adverse symptoms with the goal of reducing allergic reactions, intolerance, and sensitization. The degree of hydrolysis—the extent to which peptide bonds are broken by a hydrolysis method—may be about 3% to about 25%, or about 5% to about 15%. The proteins may be hydrolyzed by any method known in the art. In certain embodiments, the partially hydrolyzed proteins may be whey protein hydrolysate (WPH) protein (N×6.38). WPH protein generally has a size of about 3 to 10 kDa, which is small and more easily digested.

The protein component of a formulation of the present invention may comprise proteins that are small in size and are intact, i.e., are not hydrolyzed. For example, the protein component may comprise alpha-lactalbumin-enriched whey protein concentrate or isolate (“alpha-lactalbumin” is used to denote the alpha-lactalbumin protein in the formulation provided by the concentrate or isolate). Alpha-lactalbumin is a small protein (14 kDa) that is nearly the size of a hydrolyzed protein. It has been demonstrated to reduce gastrointestinal events and has a high content of essential amino acids, which enables having a lower protein formulation. Further, peptides generated from proteolysis of alpha-lactalbumin have shown in vivo to have bactericidal, opioid agonist, and immunostimulating activity.

The protein component of a formulation of the present invention may comprise other digestion-aiding proteins, including but not limited to, x-casein, bile salt-stimulated lipase, and amylase.

In embodiments of the invention, the protein component may comprise WPH protein and alpha-lactalbumin. WPH protein and alpha-lactalbumin may be present in the formulation in varying ratios, such as a ratio of about 4:1 to about 1:1 by weight, or a ratio of about 7:2 to about 5:4 by weight.

The amount of WPH protein and alpha-lactalbumin in the protein component may be presented as a percentage of the total amount of protein in the protein component. For example, WPH protein and the alpha-lactalbumin may comprise about 15% to about 95% by weight of the protein component, or about 30% to about 70%, or about 60% to about 90%, or about 60% to about 75%, or about 75% to about 90%, by weight of the protein component. In some embodiments, the WPH protein and alpha-lactalbumin may comprise about 40%, or about 44%, or about 49%, or about 53%, or about 59%, or about 63%, or about 67%, or about 70%, or about 76%, or about 83%, or about 88%, by weight of the protein component. In certain embodiments, WPH protein and alpha-lactalbumin may comprise 39.5%, 43.8%, 48.6%, 52.9%, 58.6%, or 62.9% by weight of the protein component.

WPH protein may comprise about 10% to about 75% by weight of the protein component, or about 20% to about 50%, or about 45% to about 70%, or about 45% to about 60%, or about 55% to about 70%, by weight of the protein component. In some embodiments, WPH protein may comprise about 26%, or about 35%, or about 45%, or about 49%, or about 52%, or about 55%, or about 59%, or about 65%, or about 69%, by weight of the protein component. In certain embodiments, WPH protein may comprise 25.9%, 35%, or 45% by weight of the protein component.

Alpha-lactalbumin may comprise about 5% to about 25% by weight of the protein component, or about 10% to about 20%, or about 12% to about 20%, or about 13% to about 16%, or about 15% to about 20%, by weight of the protein component. In some embodiments, alpha-lactalbumin may comprise about 14%, or about 15%, or about 17%, or about 18%, or about 19%, by weight of the protein component. In certain embodiments, alpha-lactalbumin may comprise 13.6% or 17.9% by weight of the protein component.

The amount of WPH protein and alpha-lactalbumin may be presented as grams per liter (g/L) of the formulation in embodiments in which the formulation is in liquid ready-to-feed or as-fed form (see “Methods of Administration” section below). For example, WPH protein and alpha-lactalbumin may comprise about 2 g/L to about 20 g/L, or about 4 g/L to about 15 g/L, of the formulation. In some embodiments, WPH protein and alpha-lactalbumin may comprise about 5 g/L, or about 6 g/L, or about 7 g/L, or about 8 g/L, or about 9 g/L, or about 10 g/L, or about 11 g/L, or about 12 g/L, or about 13 g/L, of the formulation. In certain embodiments, WPH protein and alpha-lactalbumin may comprise 5.4 g/L, 5.9 g/L, 6.6 g/L, 7.1 g/L, 7.9 g/L, 8.4 g/L, 8.8 g/L, 9.7 g/L, 10.8 g/L, 11.7 g/L, or 12.9 g/L, of the formulation. WPH protein may comprise about 1 g/L to about 13 g/L, or about 3 g/L to about 11 g/L, of the formulation. In some embodiments, WPH protein may comprise about 3 g/L, or about 4 g/L, or about 5 g/L, or about 6 g/L, or about 8 g/L, or about 10 g/L, of the formulation. In certain embodiments, WPH protein may comprise 3.5 g/L, 3.7 g/L, 4.7 g/L, 5.7 g/L, 6 g/L, 7.7 g/L, or 9.9 g/L, of the formulation. Alpha-lactalbumin may comprise about 0.5 g/L to about 6 g/L, or about 1 g/L to about 4 g/L, of the formulation. In some embodiments, alpha-lactalbumin may comprise about 1 g/L, or about 2 g/L, or about 3 g/L of the formulation. In certain embodiments, alpha-lactalbumin may comprise 1 g/L, 2.4 g/L, or 3 g/L of the formulation.

The protein component of a formulation of the present invention may comprise proteins in addition to the digestion-aiding proteins described above. For example, the protein component may comprise lactoferrin, osteopontin, β-casein, κ-casein, haptocorrin, lysozyme, secretory IgA, bile-salt stimulated lipase, or a combination thereof. Further additional proteins are disclosed in Bardanzellu et al. (“‘Omics’ in human colostrum and mature milk: looking to old data with new eyes,” Nutrients, 2017, vol. 9, no. 8, E843), which is incorporated herein by reference.

The protein component may comprise about 5% to about 20%, or about 8% to about 16%, by weight of the formulation. In some embodiments, the protein component may comprise about 10% or about 15% by weight of the formulation. In certain embodiments, the protein component may comprise 10% or 14.7% by weight of the formulation. Moreover, the protein component may comprise about 5 g/L to about 30 g/L, or about 10 g/L to 25 g/L, of the formulation. In some embodiments, the protein component may comprise about 13 g/L or about 22 g/L of the formulation. In certain embodiments, the protein component may comprise 13.4 g/L or 22 g/L of the formulation.

### Combination of Immunoprotective Proteins

Another aspect of the invention relates to a formulation comprising a protein component, in which the protein component comprises two or more immunoprotective proteins. An immunoprotective protein is a protein that promotes the immune system, such as having anti-viral, antibacterial, immunomodulatory, and/or anti-inflammatory effects.

The protein component of a formulation of the present invention may comprise lactoferrin. Lactoferrin is an iron-binding glycoprotein that has been proposed to play a role in iron uptake by the intestinal mucosa and to act as a bacteriostatic agent by withholding iron from iron-requiring bacteria. It is also present in neutrophils and is released during inflammation, which suggests that lactoferrin is involved in the immune response. Lactoferrin may function also as a growth factor and/or a bactericidal agent, and may promote maturation of the infant gut.

Lactoferrin may be, for example, isolated from the milk of a non-human animal or produced by a genetically modified organism. The process generally involves an absorbing step to obtain lactoferrin from raw milk material using a weakly acidic cationic exchanger, a washing step to remove nonabsorbed substances, and a desorbing step to obtain purified lactoferrin. Varying methods of producing lactoferrin are disclosed in U.S. Pat. Nos. 4,791,193, 5,849,885, 5,861,491, 5,919,913, and 7,368,141, which are all incorporated herein by reference.

The protein component of a formulation of the present invention may comprise osteopontin. Osteopontin is a multifunctional protein present in most tissues and body fluids, with the highest concentrations found in breast milk. It is thought to be involved in cell-mediated immune response, chemotaxis of inflammatory cells, anti-inflammatory responses, induction of T-helper type 1 (Th1)-type immunity, and enhanced host defense against pathogens.

The protein component may comprise other immunoprotective proteins, including but not limited to k-casein, haptocorrin, lysozyme, secretory IgA, lactoperoxidase, and bile-salt stimulated lipase. Additional proteins that relate to immune system processes and that may be included in the protein component are disclosed in Bardanzellu et al., which is incorporated herein by reference.

In embodiments of the invention, the protein component may comprise lactoferrin and osteopontin. Without being bound by theory, it is believed that the effects of lactoferrin and osteopontin may be synergistic, as each protein acts through different mechanisms. Lactoferrin works through iron sequestration to inhibit iron uptake by iron-requiring pathogens, while osteopontin is involved in systemic immunity, working on immune cells themselves and impacting gene expression and cytokines. The systemic immunity combined with a more favorable microbiome could support synergistic immune function. Further, lactoferrin and osteopontin can bind together with high affinity to form a lactoferrin-osteopontin complex, in which multiple cationic lactoferrin molecules bind to one anionic molecule of osteopontin. As shown in Example 3 below, the lactoferrin-osteopontin complex does form in an infant formula and is relatively resistant to in vitro digestion, can promote intestinal cell proliferation and differentiation, inhibit growth of enteropathogenic Escherichia coli, and increase intestinal immunity. Notably, Example 3 demonstrates that these effects are greater when associated with the lactoferrin-osteopontin complex, as compared to lactoferrin and osteopontin alone.

Lactoferrin and osteopontin may be present in the formulation in varying ratios, such as a ratio of about 12:1 to about 2:1, or about 9:1 to about 6:1, or about 11:1 to about 3:1, by weight. In some embodiments, the lactoferrin and osteopontin are present in a molecular ratio of about 10:1 to about 2:1, or about 8:1 to about 3:1, such as about 8:1, or about 5:1, or about 3:1.

Lactoferrin and osteopontin may comprise about 4% to about 35% by weight of the protein component, or about 6% to about 9%, or about 5% to about 30%, or about 8% to about 20%, or about 10% to about 25%, by weight of the protein component of the formulation. In some embodiments, lactoferrin and osteopontin may comprise about 7% to about 8%, or about 10%, or about 12%, or about 14%, or about 17%, or about 20%, or about 24%, by weight of the protein component of the formulation. In certain embodiments, lactoferrin and osteopontin may comprise 7.3% or 7.6% by weight of the protein component of the formulation. Lactoferrin may comprise about 4% to about 30%, or about 6% to about 7%, or about 5% to about 25%, or about 6% to about 20%, or about 8% to about 22%, by weight of the protein component. In some embodiments, lactoferrin may comprise about 6%, or about 7%, or about 10%, or about 12%, or about 15%, or about 18%, or about 22%, by weight of the protein component. In certain embodiments, lactoferrin may comprise 6.4% or 6.8% by weight of the protein component. Osteopontin may comprise about 0.1% to about 4%, or about 0.5% to about 1.5%, or about 1% to about 3%, or about 1.5% to about 2%, or about 2% to about 2.5%, by weight of the protein component. In some embodiments, osteopontin may comprise about 1%, or about 1.5%, or about 2%, or about 2.5%, by weight of the protein component. In certain embodiments, osteopontin may comprise 0.77% or 0.98% by weight of the protein component.

In addition, the amount of lactoferrin and osteopontin in the protein component may comprise about 0.4 g/L to about 4 g/L, or about 0.5 g/L to about 3 g/L, of the formulation. In some embodiments, lactoferrin and osteopontin may comprise about 0.6 g/L, or about 1 g/L, or about 1.5 g/L, or about 2 g/L of the formulation. In certain embodiments, lactoferrin and osteopontin may comprise 0.98 g/L or 1.67 g/L of the formulation. Lactoferrin may comprise about 0.3 g/L to about 3 g/L, or about 0.5 g/L to about 2 g/L, of the formulation. In some embodiments, lactoferrin may comprise about 0.5 g/L, or about 0.8 g/L, or about 0.9 g/L, or about 1.4 g/L, or about 1.5 g/L of the formulation. In certain embodiments, lactoferrin may comprise 0.85 g/L or 1.5 g/L of the formulation. Osteopontin may comprise about 0.05 g/L to about 0.5 g/L, or about 0.08 g/L to about 0.3 g/L, of the formulation. In some embodiments, osteopontin may comprise about 0.1 g/L or about 0.2 g/L of the formulation. In particular embodiments, osteopontin may comprise 0.13 g/L or 0.17 g/L of the formulation.

Notably, in embodiments in which the formulation is applied to infant formulas, the quantities of lactoferrin and osteopontin described herein are not found in typical infant formulas. It is believed that the levels of lactoferrin and osteopontin provide the present formulation with greater immunoprotective properties as compared to infant formulas that are currently on the market. And as further discussed below, in certain embodiments, the formulation contains non-human-derived milk and its components.

The protein component of a formulation of the present invention may comprise proteins in addition to the immunoprotective proteins described above. For example, the protein component may comprise I3-casein, x-casein, haptocorrin, lysozyme, secretory IgA, bile-salt stimulated lipase, or a combination thereof. Additional contemplated proteins are disclosed in Bardanzellu et al., which is incorporated herein by reference.

The protein component may comprise about 5% to about 20%, or about 8% to about 16%, by weight of the formulation. In some embodiments, the protein component may comprise about 10% or about 15% by weight of the formulation. In certain embodiments, the protein component may comprise 10% or 14.7% by weight of the formulation. Moreover, the protein component may comprise about 5 g/L to about 30 g/L, or about 10 g/L to about 25 g/L, of the formulation. In some embodiments, the protein component may comprise about 13 g/L or about 22 g/L of the formulation. In certain embodiments, the protein component may comprise 13.4 g/L or 22 g/L of the formulation.

### Combination of Digestion-Aiding Proteins and Immunoprotective Proteins

Yet another aspect of the invention relates to a protein component of the formulation comprising one or more digestion-aiding proteins and one or more immunoprotective proteins. Such a formulation is designed to provide both comfort and immunity, which is in contrast to many known infant formulas that are designed to address either comfort or immunity—not both. For example, infant formulas that focus on providing digestive comfort may comprise 100% hydrolyzed proteins, and therefore lack many of the other types of proteins that infants would otherwise receive through breast milk. On the other hand, infant formulas that focus on providing immunity may comprise only intact proteins and as a result may not be well-tolerated for digestion.

In some embodiments, the one or more digestion-aiding proteins and the one or more immunoprotective proteins in the formulation are present in a ratio of about 12:1 to about 1:1, or about 10:1 to about 3:1, or about 8:1 to about 3:1, or about 10:1 to about 4:1, by weight.

In embodiments of the invention, the protein component of the formulation may comprise WPH protein and lactoferrin. WPH protein and lactoferrin may be present in the formulation in varying ratios, such as a ratio of about 9:1 to about 1:1, or about 8:1 to about 2:1, or about 7:1 to about 5:2, by weight.

WPH protein and lactoferrin may comprise about 10% to about 95% by weight of the protein component, or about 25% to about 60%, or about 60% to about 85%, or about 60% to about 70%, or about 75% to about 85%, by weight of the protein component of the formulation. In some embodiments, WPH protein and lactoferrin may comprise about 32%, or about 41%, or about 51%, or about 62%, or about 64%, or about 67%, or about 78%, or about 80%, or about 81%, by weight of the protein component. In certain embodiments, WPH protein and lactoferrin may comprise 32.4%, 32.8%, 41.4%, 41.8%, 51.4%, or 51.8%, by weight of the protein component. WPH protein may comprise about 10% to about 75% by weight of the protein component, or about 20% to about 50%, or about 45% to about 70%, or about 45% to about 60%, or about 55% to about 70%, by weight of the protein component. In some embodiments, WPH protein may comprise about 26%, or about 35%, or about 45%, or about 49%, or about 52%, or about 55%, or about 59%, or about 65%, or about 69%, by weight of the protein component. In certain embodiments, WPH protein may comprise 25.9%, or 35%, or 45% by weight of the protein component. Lactoferrin may comprise about 4% to about 30% by weight of the protein component, or about 6% to about 7%, or about 5% to about 25%, or about 6% to about 20%, or about 8% to about 22%, by weight of the protein component. In some embodiments, lactoferrin may comprise about 6% or about 7% by weight of the protein component. In certain embodiments, lactoferrin may comprise 6.4% or 6.8% by weight of the protein component.

Further, the amount of WPH protein and lactoferrin in the protein component of the formulation may comprise about 2 g/L to about 15 g/L, or about 3 g/L to about 14 g/L, of the formulation. In some embodiments, WPH protein and lactoferrin may comprise about 4 g/L, or about 5 g/L, or about 6 g/L, or about 7 g/L, or about 9 g/L, or about 11 g/L, or about 12 g/L, of the formulation. In certain embodiments, WPH protein and lactoferrin may comprise 4.35 g/L, 5 g/L, 5.55 g/L, 6.2 g/L, 6.55 g/L, 6.85 g/L, 8.55 g/L, 9.2 g/L, 10.8 g/L, or 11.4 g/L, of the formulation. WPH protein may comprise about 1 g/L to about 13 g/L, or about 3 g/L to about 11 g/L, of the formulation. In some embodiments, WPH protein may comprise about 3 g/L, or about 4 g/L, or about 5 g/L, or about 6 g/L, or about 8 g/L, or about 10 g/L, of the formulation. In certain embodiments, WPH protein may comprise 3.5 g/L, 3.7 g/L, 4.7 g/L, 5.7 g/L, 6 g/L, 7.7 g/L, or 9.9 g/L, of the formulation. Lactoferrin may comprise about 0.3 g/L to about 3 g/L, or about 0.5 g/L to about 2 g/L, of the formulation. In some embodiments, lactoferrin may comprise about 0.5 g/L, or about 0.8 g/L, or about 0.9 g/L, or about 1.4 g/L, or about 1.5 g/L of the formulation. In certain embodiments, lactoferrin may comprise 0.85 g/L or 1.5 g/L of the formulation.

In embodiments of the invention, the protein component of the formulation may comprise WPH protein and osteopontin. WPH protein and osteopontin may be present in the formulation in varying ratios, such as a ratio of about 60:1 to about 10:1 by weight, or a ratio of about 50:1 to about 25:1 by weight.

WPH protein and osteopontin may comprise about 10% to and about 80% by weight of the protein component, or about 20% to about 55%, or about 45% to about 75%, or about 45% to about 65%, or about 55% to about 75%, by weight of the protein component of the formulation. In some embodiments, WPH protein and osteopontin may comprise about 27%, or about 36%, or about 46% by weight of the protein component. In certain embodiments, WPH protein and osteopontin may comprise 26.67%, 26.88%, 35.77%, 35.98%, 45.77%, or 45.98% by weight of the protein component. WPH protein may comprise about 10% to about 75%, or about 20% to about 50%, or about 45% to about 70%, or about 45% to about 60%, or about 55% to about 70%, by weight of the protein component. In some embodiments, WPH protein may comprise about 26%, or about 35%, or about 45%, or about 49%, or about 52%, or about 55%, or about 59%, or about 65%, or about 69%, by weight of the protein component. In certain embodiments, WPH protein may comprise 25.9%, 35%, or 45% by weight of the protein component. Osteopontin may comprise about 0.1% to about 4%, or about 0.5% to about 1.5%, or about 1% to about 3%, or about 1.5% to about 2%, or about 2% to about 2.5%, by weight of the protein component. In some embodiments, osteopontin may comprise about 1%, or about 1.5%, or about 2%, or about 2.5%, by weight of the protein component. In certain embodiments, osteopontin may comprise 0.77% or 0.98% by weight of the protein component.

In addition, the amount of WPH protein and osteopontin in the formulation may comprise about 1 g/L to about 14 g/L, or about 3 g/L to about 12 g/L, of the formulation. In some embodiments, WPH protein and osteopontin may comprise about 3 g/L, or about 4 g/L, or about 5 g/L, or about 6 g/L, or about 8 g/L, or about 10 g/L, of the formulation. In certain embodiments, WPH protein and osteopontin may comprise 3.63 g/L, 3.67 g/L, 3.83 g/L, 4.83 g/L, 4.87 g/L, 5.83 g/L, 5.87 g/L, 6.13 g/L, 6.17 g/L, 7.83 g/L, 7.87 g/L, 10.03 g/L, or 10.07 g/L, of the formulation. WPH protein may comprise about 1 g/L to about 13 g/L, or about 3 g/L to about 11 g/L, of the formulation. In some embodiments, WPH protein may comprise about 3 g/L, or about 4 g/L, or about 5 g/L, or about 6 g/L, or about 8 g/L, or about 10 g/L, of the formulation. In certain embodiments, WPH protein may comprise 3.5 g/L, 3.7 g/L, 4.7 g/L, 5.7 g/L, 6 g/L, 7.7 g/L, or 9.9 g/L, of the formulation. Osteopontin may comprise about 0.05 g/L to about 0.5 g/L, or about 0.08 g/L to about 0.3 g/L, of the formulation. In some embodiments, osteopontin may comprise about 0.1 g/L or about 0.2 g/L of the formulation. In particular embodiments, osteopontin may comprise 0.13 g/L or 0.17 g/L of the formulation.

In embodiments of the invention, the protein component of the formulation may comprise alpha-lactalbumin and lactoferrin. Alpha-lactalbumin and lactoferrin may be present in the formulation in varying ratios, such as a ratio of about 4:1 to about 0.5:1 by weight, or a ratio of about 3:1 to about 2:1, or about 2:1 to about 0.8:1, by weight.

Alpha-lactalbumin and lactoferrin may comprise about 5% to about 55% by weight of the protein component, or about 15% to about 30%, or about 15% to about 45%, or about 20% to about 35%, or about 20% to about 40%, by weight of the protein component of the formulation. In some embodiments, alpha-lactalbumin and lactoferrin may comprise about 20% or about 25% by weight of the protein component. In certain embodiments, alpha-lactalbumin and lactoferrin may comprise about 20% or about 24.7% by weight of the protein component. Alpha-lactalbumin may comprise about 5% to about 25%, or about 10% to about 20%, or about 12% to about 20%, or about 13% to about 16%, or about 15% to about 20%, by weight of the protein component. In some embodiments, the alpha-lactalbumin may comprise about 14%, or about 15%, or about 17%, or about 18%, or about 19%, by weight of the protein component. In certain embodiments, alpha-lactalbumin may comprise 13.6% or 17.9% by weight of the protein component. Lactoferrin may comprise about 4% to about 30%, or about 6% to about 7%, or about 5% to about 25%, or about 6% to about 20%, or about 8% to about 22%, by weight of the protein component. In some embodiments, lactoferrin may comprise about 6%, or about 7%, or about 10%, or about 12%, or about 15%, or about 18%, or about 22%, by weight of the protein component. In certain embodiments, lactoferrin may comprise 6.4% or 6.8% by weight of the protein component.

Further, the amount of alpha-lactalbumin and lactoferrin may comprise about 0.9 g/L to about 9 g/L, or about 1 g/L to about 6 g/L, or about 2 g/L to about 5 g/L, of the formulation. In some embodiments, alpha-lactalbumin and lactoferrin may comprise about 3 g/L, or about 4 g/L, or about 5 g/L of the formulation. In certain embodiments, alpha-lactalbumin and lactoferrin may comprise 3.25 g/L or 4.5 g/L of the formulation. Alpha-lactalbumin may comprise about 0.5 g/L to about 6 g/L, or about 1 g/L to about 4 g/L, of the formulation. In some embodiments, alpha-lactalbumin may comprise about 1 g/L, or about 2 g/L, or about 3 g/L of the formulation. In certain embodiments, alpha-lactalbumin may comprise 2.4 g/L or 3 g/L of the formulation. Lactoferrin may comprise about 0.3 g/L to about 3 g/L, or about 0.5 g/L to about 2 g/L, of the formulation. In some embodiments, lactoferrin may comprise about 0.5 g/L, or about 0.8 g/L, or about 0.9 g/L, or about 1.4 g/L, or about 1.5 g/L of the formulation. In certain embodiments, lactoferrin may comprise 0.85 g/L or 1.5 g/L of the formulation.

In embodiments of the invention, the protein component of the formulation may comprise alpha-lactalbumin and osteopontin. Alpha-lactalbumin and osteopontin may be present in the formulation in varying ratios, such as a ratio of about 25:1 to about 5:1 by weight, or a ratio of about 20:1 to about 7:1 by weight.

Alpha-lactalbumin and osteopontin may comprise about 5% to about 30% by weight of the protein component, or about 10% to about 25%, or about 13% to about 23%, or about 14% to about 18%, or about 17% to about 23%, by weight of the protein component of the formulation. In some embodiments, alpha-lactalbumin and osteopontin may comprise about 14% or about 19% by weight of the protein component. In certain embodiments, alpha-lactalbumin and osteopontin may comprise 14.37% or 18.88% by weight of the protein component. Alpha-lactalbumin may comprise about 5% to about 25%, or about 10% to about 20%, or about 12% to about 20%, or about 13% to about 16%, or about 15% to about 20%, by weight of the protein component. In some embodiments, the alpha-lactalbumin may comprise about 14%, or about 15%, or about 17%, or about 18%, or about 19%, by weight of the protein component. In certain embodiments, alpha-lactalbumin may comprise 13.6% or 17.9% by weight of the protein component. Osteopontin may comprise about 0.1% to about 4%, or about 0.5% to about 1.5%, or about 1% to about 3%, or about 1.5% to about 2%, or about 2% to about 2.5%, by weight of the protein component. In some embodiments, osteopontin may comprise about 1%, or about 1.5%, or about 2%, or about 2.5%, by weight of the protein component. In certain embodiments, osteopontin may comprise 0.77% or 0.98% by weight of the protein component.

Further, the amount of alpha-lactalbumin and osteopontin may comprise about 0.5 g/L to about 7 g/L, or about 1 g/L to about 5 g/L, of the formulation. In some embodiments, alpha-lactalbumin and osteopontin may comprise about 2 g/L or about 3 g/L of the formulation. In certain embodiments, alpha-lactalbumin and osteopontin may comprise 2.53 g/L or 3.17 g/L of the formulation. Alpha-lactalbumin may comprise about 0.5 g/L to about 6 g/L, or about 1 g/L to about 4 g/L, of the formulation. In some embodiments, alpha-lactalbumin may comprise about 1 g/L, or about 2 g/L, or about 3 g/L of the formulation. In certain embodiments, alpha-lactalbumin may comprise 2.4 g/L or 3 g/L of the formulation. Osteopontin may comprise about 0.05 g/L to about 0.5 g/L, or about 0.08 g/L to about 0.3 g/L, of the formulation. In certain embodiments, osteopontin may comprise about 0.1 g/L or about 2 g/L of the formulation. In particular embodiments, osteopontin may comprise 0.13 g/L or 0.17 g/L of the formulation.

In addition, the protein component of a formulation of the present invention may comprise a combination of WPH protein, lactoferrin, and osteopontin; or a combination of alpha-lactalbumin, lactoferrin, and osteopontin; or a combination of WPH protein, alpha-lactalbumin, and lactoferrin; or a combination of WPH protein, alpha-lactalbumin, and osteopontin.

In embodiments of the invention, the protein component may comprise WPH protein, alpha-lactalbumin, lactoferrin, and osteopontin. WPH protein, alpha-lactalbumin, lactoferrin, and osteopontin may comprise about 15% to about 95%, or about 45% to about 80%, by weight of the protein component. In certain embodiments, WPH protein, alpha-lactalbumin, lactoferrin, and osteopontin may comprise about 50% to about 75% of the protein component. WPH protein may comprise about 10% to about 75% by weight of the protein component, or about 20% to about 60% by weight of the protein component; alpha-lactalbumin may comprise about 5% to about 25% by weight of the protein component, or about 10% to about 20% by weight of the protein component; lactoferrin may comprise about 4% to about 30% by weight of the protein component, or about 6% to about 20% by weight of the protein component; and osteopontin may comprise about 0.1% to about 4% by weight of the protein component, or about 0.5% to about 2% by weight of the protein component. In some embodiments, WPH protein may comprise about 26%, or about 35%, or about 45% by weight of the protein component; alpha-lactalbumin may comprise about 14% or about 18% by weight of the protein component; lactoferrin may comprise about 6% or about 7% by weight of the protein component; and osteopontin may comprise about 1% by weight of the protein component. In certain embodiments, WPH protein may comprise 25.9% or 35% or 45% by weight of the protein component; alpha-lactalbumin may comprise 13.6% or 17.9% by weight of the protein component; lactoferrin may comprise 6.4% or 6.8% by weight of the protein component; and osteopontin may comprise 0.77% or 0.98% by weight of the protein component.

Further, the amount of WPH protein may comprise about 1 g/L to about 13 g/L, or about 3 g/L to about 11 g/L, of the formulation; the amount of alpha-lactalbumin may comprise about 0.5 g/L to about 6 g/L, or about 1 g/L to about 4 g/L, of the formulation; the amount of lactoferrin may comprise about 0.3 g/L to about 3 g/L, or about 0.5 g/L to about 2 g/L, of the formulation; and the amount of osteopontin may comprise about 0.05 g/L to about 0.5 g/L, or about 0.08 g/L to about 0.3 g/L. In some embodiments, WPH protein may comprise about 3 g/L, or about 5 g/L, or about 6 g/L, or about 8 g/L, or about 10 g/L, of the formulation; alpha-lactalbumin may comprise about 2 g/L or about 3 g/L of the formulation; lactoferrin may comprise about 0.9 g/L or about 1.5 g/L of the formulation; and osteopontin may comprise about 0.1 g/L or about 0.2 g/L of the formulation. In certain embodiments, WPH protein may comprise 3.5 g/L, 4.7 g/L, or 5.7 g/L, or 6 g/L, or 7.7 g/L, or 9.9 g/L, of the formulation; alpha-lactalbumin may comprise 2.4 g/L or 3 g/L of the formulation; lactoferrin may comprise 0.85 g/L or 1.5 g/L of the formulation; and osteopontin may comprise about 0.13 g/L or about 0.17 g/L of the formulation.

In embodiments of the invention, the protein component may comprise WPH protein, alpha-lactalbumin, lactoferrin, and osteopontin, in which the combination of WPH protein and alpha-lactalbumin may be present in a ratio to the combination of lactoferrin and osteopontin of about 12:1 to about 1:1 or about 10:1 to about 3:1, by weight. Examples include about 8:1 or about 5:1 or about 3:1 by weight. The combination of WPH protein and alpha-lactalbumin may be present in a ratio to the lactoferrin of about 13:1 to about 1:1, or about 10:1 to about 2:1 or about 9:1 to about 3:1, by weight. Examples include about 9:1 or about 6:1 or about 4:1 by weight. The combination of WPH protein and alpha-lactalbumin may be present in a ratio to the osteopontin of about 45:1 to about 25:1, or about 40:1 to about 30:1 or about 38:1 to about 34:1, by weight. Examples include about 36:1 by weight.

In embodiments of the invention, the protein component may comprise WPH protein, alpha-lactalbumin, and lactoferrin. WPH protein, alpha-lactalbumin, and lactoferrin may comprise about 15% to about 95%, or about 45% to about 80%, by weight of the protein component. In certain embodiments, WPH protein, alpha-lactalbumin, and lactoferrin may comprise about 50% to about 75% of the protein component. WPH protein may comprise about 10% to about 75% by weight of the protein component, or about 20% to about 60% by weight of the protein component; alpha-lactalbumin may comprise about 5% to about 25% by weight of the protein component, or about 10% to about 20% by weight of the protein component; and lactoferrin may comprise about 4% to about 30% by weight of the protein component, or about 6% to about 20% by weight of the protein component

The protein component of a formulation of the present invention may comprise proteins in addition to the digestion-aiding proteins and immunoprotective proteins described above. Further additional proteins are disclosed in Bardanzellu et al., which is incorporated herein by reference.

The protein component may comprise about 5% to about 20%, or about 8% to about 16%, by weight of the formulation. In some embodiments, the protein component may comprise about 10% or about 15% by weight of the formulation. In certain embodiments, the protein component may comprise 10% or 14.7% by weight of the formulation. Moreover, the protein component may comprise about 5 g/L to about 30 g/L, or about 10 g/L to about 25 g/L, of the formulation. In some embodiments, the protein component may comprise about 13 g/L or about 22 g/L of the formulation. In certain embodiments, the protein component may comprise 13.4 g/L or 22 g/L of the formulation.

### Additional Components of the Formulation

In addition to the protein component described above, the formulations of the invention may comprise other components.

For example, the formulations may comprise a fat component. The fat component may comprise, for instance, innate MFGM, added MFGM, phospholipids, cholesterol, nonhexane-extracted DHA, hexane-extracted AA, nonhexane-extracted AA, or combination thereof. Suitable fat or lipid sources for the fat component of the formulation may be of any known or used in the art, including but not limited to, animal sources, e.g., milk fat, butter, butter fat, egg yolk lipid, and SN2 palmitate oil; marine sources, such as fish oils, marine oils, single cell oils; vegetable and plant oils, such as corn oil, canola oil, sunflower oil, soybean oil, palm oil, palm olein oil, coconut oil, high oleic sunflower oil, safflower oil, high-oleic safflower oil, evening primrose oil, rapeseed oil, olive oil, flaxseed (linseed) oil, cottonseed oil, palm stearin oil, palm kernel oil, wheat germ oil; medium chain triglyceride oils and emulsions and esters of fatty acids; and any combinations thereof.

The fat component may comprise about 15% to about 40%, or about 20% to about 35%, by weight of the formulation. In certain embodiments, the fat component may comprise about 28% by weight of the formulation. Moreover, the fat component may comprise about 25 g/L to about 50 g/L, or about 30 g/L to about 45 g/L, or about 35 g/L to about 40 g/L, of the formulation. In certain embodiments, the fat component may comprise about 37 g/L, including 37.3 g/L, of the formulation.

In certain embodiments, the fat component comprises MFGM. MFGM comprises milk fat globules (MFGs) in a bioactive membrane system, and makes up the fat in bovine milk. Fat is the second largest constituent of bovine milk dry matter having nutritional significance, and MFGM as a whole or some of its associated individual components have physiological and nutritional functions, including supporting antiviral and antibacterial mechanisms that combat gut-derived infections.

In embodiments of the invention, the formulation may comprise osteopontin and MFGM. Such a formulation may be effective in providing immunoprotection, as osteopontin may have a systemic immunity mechanism of action, while MFGM may be acting on mucosal local immunity.

In certain embodiments, the fat component comprises AA. AA may comprise about 0.1% to about 1.2% by weight of the fat component, or about 0.5% to about 0.8% by weight of the fat component. In certain embodiments, AA may comprise about 0.6% of the fat component. In addition, AA may comprise about 0.1 g/L to about 0.2 g/L, or about 0.15 g/L to about 0.18 g/L, of the formulation. In particular embodiments, AA may comprise about 0.17 g/L of the formulation.

In certain embodiments, the fat component comprises DHA. DHA may comprise about 0.05% to about 0.5% by weight of the fat component, or about 0.2% to about 0.4% by weight of the fat component. In certain embodiments, DHA may comprise about 0.3%, or about 0.38%, of the fat component. Further, DHA may comprise about 0.05 g/L to about 0.3 g/L, or about 0.08 g/L to about 0.15 g/L, of the formulation. In particular embodiments, DHA may comprise about 0.11 g/L, or about 0.141 g/L, of the formulation. Moreover, DHA may comprise about 10 mg/100 cal to about 50 mg/100 cal, or about 20 mg/100 cal to about 40 mg/100 cal. In certain embodiments, DHA may comprise about 16 mg/100 cal, or about 20 mg/100 cal, or about 32 mg/100 cal, or about 40 mg/100 cal.

In certain embodiments, the fat component comprises a combination of low erucic acid rapeseed oil (canola oil) in an amount of about 10% to about 40%, or about 15% to about 35%, or about 20% to about 30%, or about 25%, or 25.3%, by weight of the fat component; sunflower oil in an amount of about 1% to about 15%, or about 4% to about 12%, or about 6% to about 10%, or about 8%, or 8.4%, by weight of the fat component; high oleic sunflower seed oil in an amount of about 20% to about 50%, or about 25% to about 45%, or about 30% to about 40%, or about 35%, or about 34%, or 33.8%, by weight of the fat component; coconut oil in an amount of about 5% to about 30%, or about 10% to about 25%, or about 12% to about 20%, or about 15%, or about 17%, or 16.9%, by weight of the fat component; an oil source for AA, such as ARASCO® oil, in an amount of about 0.1% to about 8%, or about 0.5% to about 6%, or about 1% to about 4%, or about 2%, or about 1%, or about 1.5%, or 1.54%, by weight of the fat component; an oil source for DHA, such as DHASCO® or DHASCO-B® oil, in an amount of about 0.1% to about 4%, or about 0.3% to about 2%, or about 0.5% to about 1.5%, or about 1%, or 0.99%, by weight of the fat component; and monoglycerides, such as monoglyceryl palmitate, monoglyceryl stearate, monoglyceryl oleate, monoglyceryl linoleate, and combinations thereof, in an amount that provides about 0.1% to about 5%, or about 0.5% to about 2%, or about 1%, or 0.9%, by weight of the fat component.

In certain embodiments, the fat component comprises a combination of canola oil in an amount of about 1 g/L to about 20 g/L, or about 5 g/L to about 15 g/L, or about 8 g/L to about 12 g/L, or about 10 g/L, or 9.77 g/L; sunflower oil in an amount of about 1 g/L to about 6 g/L, or about 2 g/L to about 4 g/L, or about 3 g/L, or 3.26 g/L; high oleic sunflower seed oil in an amount of about 5 g/L to about 20 g/L, or about 8 g/L to about 18 g/L, or about 10 g/L to about 15 g/L, or about 13 g/L, or 13.02 g/L; coconut oil in an amount of about 1 g/L to about 15 g/L, or about 3 g/L to about 10 g/L, or about 5 g/L to about 8 g/L, or about 6 g/L, or about 7 g/L, or 6.51 g/L; an oil source for AA, such as ARASCO® oil, in an amount of about 0.1 g/L to about 3 g/L, or about 0.2 g/L to about 2 g/L, or about 1 g/L, or about 0.6 g/L, or about 0.5 g/L, or 0.59 g/L; an oil source for DHA, such as DHASCO® or DHASCO-B® oil, in an amount of about 0.1 g/L to about 2 g/L, or about 0.2 g/L to about 1 g/L, about 0.3 g/L, or about 0.4 g/L, or 0.38 g/L; and monoglycerides, such as monoglyceryl palmitate, monoglyceryl stearate, monoglyceryl oleate, monoglyceryl linoleate, and combinations thereof, in an amount of about 0.2 g/L to about 1 g/L, or about 0.25 g/L to about 0.4 g/L, about 0.3 g/L, or about 0.35 g/L, or 0.33 g/L.

In certain embodiments, the fat component comprises a combination of canola oil in an amount of about 20% to about 40%, or about 25% to about 35%, or about 28% to about 32%, or about 30%, or 29.7%, by weight of the fat component; sunflower oil in an amount of about 1% to about 20%, or about 5% to about 15%, or about 8% to about 12%, or about 10%, or 9.9%, by weight of the fat component; high oleic sunflower seed oil in an amount of about 30% to about 50%, or about 35% to about 45%, or about 38% to about 42%, or about 39%, or about 40%, or 39.6%, by weight of the fat component; coconut oil in an amount of about 10% to about 30%, or about 15% to about 25%, or about 18% to about 22%, or about 19%, or about 20%, or 19.8%, by weight of the fat component; monoglycerides, such as monoglyceryl palmitate, monoglyceryl stearate, monoglyceryl oleate, monoglyceryl linoleate, and combinations thereof, in an amount that provides about 0.1% to about 2%, or about 0.5% to about 1.5%, or about 0.8% to about 1.2%, or about 1%, by weight of the fat component; tocopherols, such as RRR-tocopherols concentrate (70% concentrate in edible vegetable oil), in an amount that provides about 0.015% to about 0.04%, or about 0.02% to about 0.035%, or about 0.025%, or about 0.03%, or 0.028%, by weight of the fat component; and ascorbyl palmitate in an amount that provides about 0.001% to about 0.025%, or about 0.005% to about 0.02%, or about 0.008% to about 0.018%, or about 0.01%, or about 0.015%, or 0.014%, by weight of the fat component.

The formulations of the present invention may also comprise a carbohydrate component. The carbohydrate component may comprise, for example, lactose, glucose, fructose, galactooligosaccharide (GOS), fructooligosaccharide (FOS), inulin, corn syrup solids, dextrin, maltodextrin, sucrose, polydextrose, dextrose, tapioca, starch, tapioca starch, rice syrup solids, waxy corn, waxy rice starch, and the like.

The carbohydrate component may comprise about 40% to about 70%, or about 50% to about 60%, by weight of the formulation. In certain embodiments, the carbohydrate component may comprise about 57% by weight of the formulation. Moreover, the carbohydrate component may comprise about 60 g/L to about 90 g/L, or about 70 g/L to about 80 g/L, of the formulation. In certain embodiments, the carbohydrate component may comprise about 76 g/L of the formulation.

In certain embodiments, the formulation may comprise lactose. Lactose is thought to have some important natural prebiotic benefits, and also can provide a natural sweetness to the formulation, which avoids the need for artificial sweeteners.

In some embodiments, the formulation may comprise GOS. GOS is a prebiotic derived from lactose that has been shown to drive stool softening and other potential digestive benefits. GOS may comprise about 2% to about 10% by weight of the carbohydrate component, or about 4% to about 6% by weight of the carbohydrate component. In certain embodiments, GOS may comprise about 5% of the carbohydrate component. In addition, GOS may comprise about 1 g/L to about 8 g/L, or about 3 g/L to about 6 g/L, of the formulation. In particular embodiments, GOS may comprise about 4 g/L of the formulation.

In certain embodiments, the formulation may comprise FOS, which is also a prebiotic.

In embodiments of the invention, the formulation may comprise a milk component. The milk component may comprise milk from a non-human mammal, such as a cow, sheep, goat, yak, water buffalo, horse, reindeer, or camel. The milk may be whole milk, reduced-fat milk (2% milk fat), low-fat milk (1% milk fat), or skimmed milk. In some embodiments, the milk component may comprise non-animal milk, such as soy milk, rice milk, hemp milk, pea milk, or almond or other nut-based milks.

In certain embodiments, the milk component comprises whole milk from a bovine source. Whole milk can help the formulation to attain innate levels of MFGM, phospholipids, and cholesterol that are important, especially for infant development. Whole milk also allows less reliance on other sources of fat, such as oil, to achieve optimal nutrition.

In certain embodiments, the milk in the milk component may also be organic and/or from grass-fed animal sources. Such milk has been linked with higher levels of conjugated linoleic acid and other important vitamins and nutrients, due to the animal spending more time on pasture and consuming higher amounts of grass. Further, organic farming practices reduce exposure to environmental toxins, which are fat soluble and therefore more likely to be found in fat.

In some embodiments, milk may be present in an amount that provides about 8% to about 40%, or about 12% to about 30%, of the protein component. In some embodiments, milk may be present in an amount that provides about 1% to about 40%, or about 8% to about 35%, or about 12% to about 30%, by weight of the protein component. In certain embodiments, milk may be present in an amount that provides 15.7%, 15.9%, 17.7%, or 24.9% of the protein component. One of ordinary skill in the art would understand, based on the protein content of the milk, how much milk is necessary to provide the protein amounts set forth herein.

In some embodiments, milk may be present in an amount that provides about 5% to about 20%, or about 8% to about 15%, or about 10% to about 13%, or about 11%, or about 12%, or 11.4%, by weight of the fat component of the formulation. One of ordinary skill in the art would understand, based on the fat content of the milk, how much milk is necessary to provide the fat amount set forth herein.

Finally, the formulation may comprise various vitamins and minerals. The selection of vitamins and minerals and their quantities will vary based on the application of the formulation, as one of ordinary skill in the art would recognize. For example, an infant may have different vitamin and mineral requirements than a child of age one to thirteen years old, or an adult.

Examples of vitamins or derivations thereof may include, but are not limited to, vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, 6-carotene and any combination thereof.

Examples of minerals or derivations thereof may include, but are not limited to, boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters, and chelates of any mineral compound.

In some embodiments, the vitamins or minerals in the formulation may be provided, in part of or in full, from the milk component. In certain embodiments, the vitamins or minerals in the formulation may be provided from non-milk sources known in the art in addition to, or instead of, from the milk component.

In embodiments in which the formulation of the invention is used in infant formulas, the formulation may comprise a lower amount of iron than typically present in such products. Free iron may feed pathogenic bacteria, foster a less favorable microbiome, and potentially cause digestive discomfort. In addition, babies are born with natural stores of iron that are sufficient until around six months of age, at which point babies typically derive their daily intake of iron from introduction of solid foods and fortified foods.

Typical formulas comprise iron levels of about 1.8 mg/100 cal or 1.9 mg/100 cal, which is greater than ten times the lower limit of the FDA-mandated level of 0.15 mg/100 cal. The formulation of the invention may comprise iron levels no greater than about 1.5 mg/100 cal, or no greater than about 1.3 mg/100 cal. In some embodiments, the formulation of the invention may comprise about 1.1 mg/100 cal.

### Dosage Forms

The formulations of the present invention may be in any form suitable for administration including those known in the art, such as a powder, a gel, a suspension, a paste, a solid, a liquid, a liquid concentrate, a reconstituteable powdered milk substitute, or a ready-to-use product. For instance, the formulations may be in the form of foods, beverages, tablets, capsules and powders.

In some embodiments, the formulation may be in a form selected from the group consisting of pellets, beads, beadlets, granules, powder, or a combination thereof. In preferred embodiments, the formulation is in powder form, having a particle size in the range of about 2 μm to about 2000 μm, or in the range of about 10 μm to about 500 μm.

In certain embodiments, the formulation may be prepared as a tablet or capsule. These tablets or capsules may contain conventional excipients such as binding agents, fillers, lubricants, disintegrants, or wetting agents. In one aspect the tablets or capsules are coated according to methods well known in the art.

In embodiments in which the formulation is prepared as a tablet, the formulation may be compacted into the dosage form. The disintegrant used in such a tablet is not particularly limited, as far as it is a disintegrant used for pharmaceutical preparations. Examples can include, but are not limited to, one or more of crospovidone, crystalline cellulose, hydroxypropylcellulose with a low degree of substitution, croscarmellose sodium, carmellose calcium, carboxystarch sodium, carboxymethyl starch sodium, potato starch, wheat starch, com starch, rice starch, partly pregelatinized starch, and hydroxypropyl starch.

Examples of pharmaceutically acceptable additives used in a tablet comprising a formulation of the present invention can include excipients, lubricants, pH adjusters, taste-masking agents, sweeteners, acidifiers, refrigerants, foaming agents, preservatives, fluidizers, antioxidants, colorants, stabilizers, surfactants, buffering agents, flavors, binders and drug solubilizers. A person skilled in the art may immediately list specific examples of these additives.

Examples of a lubricant used in the tablet of the present invention can include light anhydrous silicic acid, magnesium stearate, stearic acid, calcium stearate, aluminum stearate, aluminum monostearate, sucrose fatty acid esters, polyethylene glycol, sodium stearyl fumarate, stearyl alcohol, talc, titanium oxide, hydrous silicon dioxide, magnesium silicate, synthetic aluminum silicate, calcium hydrogen phosphate, hardened castor oil, hardened rapeseed oil, Carnauba Wax, bees wax, microcrystalline wax and sodium lauryl sulfate. One or two or more lubricants can be used.

One or more hydrophilic polymers may be used in a dosage form of the invention. Examples include, but are not limited to, natural or partially or totally synthetic hydrophilic gums such as acacia, gum tragacanth, locust bean gum, guar gum, and karaya gum; cellulose derivatives such as methyl cellulose, hydroxymethyl cellulose, hydroxypropylmethyl cellulose, hydroxypropyl cellulose, hydroxyethyl cellulose, and carboxymethyl cellulose; proteinaceous substances such as agar, pectin, carrageen, and alginates; hydrophilic polymers such as carboxypolymethylene; gelatin; casein; zein; bentonite; magnesium aluminum silicate; polysaccharides; modified starch derivatives; and other hydrophilic polymers known in the art. An addition example is a carbomer, such as Carbopol 971P.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., PH102 or PH200, Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc. One or more diluents may be used in the dosage form.

In embodiments where the formulation is in a solid dosage form, e.g., a tablet, one or more binders can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate.

When the formulations are in the form of a tablet, the shape of the tablet is not particularly limited, as far as it can be produced without difficulty using an ordinary manufacturing apparatus or a manufacturing apparatus with some modifications. A disc shape that is a general concept for tablets can be mentioned as a typical example. The whole size is not particularly limited. For example, the shorter diameter (diameter for a disc tablet) is appropriately in the range of about 6 mm to about 20 mm, or about 8 mm to about 12 mm. The thickness is neither particularly limited, but appropriately about 1 mm to about 10 mm, or about 2 mm to about 8 mm.

In various embodiments, the formulations of the present invention may be in a ready-to-use composition. The ready-to-use composition may comprise one or more stabilizing agents, which include, but are not limited to, buffering agents, tonicity agents, polymers, preservatives, antioxidants, sugars and salts, and combinations thereof.

### Uses of the Formulations

An aspect of the present invention relates to different methods that involve the use of the formulations of the present invention. For example, the present invention is directed to a method of providing nutritional support to a subject. The method may comprise administering to the subject a formulation according to the present invention.

The present invention is directed to a method of promoting or inducing proliferation of intestinal cells in a subject in need thereof. The method may comprise administering to the subject a formulation according to the present invention.

The present invention is directed to a method of promoting or inducing differentiation of intestinal cells in a subject in need thereof. The method may comprise administering to the subject a formulation according to the present invention.

The present invention is directed to a method of preventing or inhibiting growth of enteropathogenic Escherichia coli (EPEC) in the digestive system of a subject in need thereof. The method may comprise administering to the subject a formulation according to the present invention.

The present invention is directed to a method of preventing or inhibiting bacterial infection in the intestinal lumen of a subject in need thereof. Such an infection may be prevented or inhibited by preventing or inhibiting the growth of bacteria. The method may comprise administering to the subject a formulation according to the present invention.

The present invention is directed to a method of increasing IL-18 secretion by intestinal cells in a subject in need thereof. The method may comprise administering to the subject a formulation according to the present invention.

The present invention is directed to a method of increasing intestinal immunity in a subject in need thereof. Such an increase in intestinal immunity may be due to an increase in the section of IL-18 by intestinal cells. The method may comprise administering to the subject a formulation according to the present invention.

An aspect of the present invention relates to different uses of the formulations of the present invention. For example, the present invention is directed to use of a formulation of the present invention to provide nutritional support to a subject. The use may comprise administering to the subject a formulation according to the present invention.

The present invention is directed to use of a formulation of the present invention to promote or induce proliferation of intestinal cells in a subject in need thereof. The use may comprise administering to the subject a formulation according to the present invention.

The present invention is directed to use of a formulation of the present invention to promote or induce differentiation of intestinal cells in a subject in need thereof. The use may comprise administering to the subject a formulation according to the present invention.

The present invention is directed to use of a formulation of the present invention to prevent or inhibit growth of EPEC in the digestive system of a subject in need thereof. The use may comprise administering to the subject a formulation according to the present invention.

The present invention is directed to use of a formulation of the present invention to prevent or inhibit bacterial infection in the intestinal lumen of a subject in need thereof. Such an infection may be prevented or inhibited by preventing or inhibiting the growth of bacteria. The use may comprise administering to the subject a formulation according to the present invention.

The present invention is directed to use of a formulation of the present invention to increase IL-18 secretion by intestinal cells in a subject in need thereof. The use may comprise administering to the subject a formulation according to the present invention.

The present invention is directed to use of a formulation of the present invention to increase intestinal immunity in a subject in need thereof. Such an increase in intestinal immunity may be due to an increase in the section of IL-18 by intestinal cells. The use may comprise administering to the subject a formulation according to the present invention.

Further, an aspect of the present invention relates to a formulation of the present invention for various uses. For example, the present invention is directed a formulation of the present invention for use in providing nutritional support to a subject. The use may comprise administering to the subject the formulation according to the present invention.

The present invention is directed a formulation of the present invention for use in promoting or inducing proliferation of intestinal cells in a subject in need thereof. The use may comprise administering to the subject the formulation according to the present invention.

The present invention is directed a formulation of the present invention for use in promoting or inducing differentiation of intestinal cells in a subject in need thereof. The use may comprise administering to the subject the formulation according to the present invention.

The present invention is directed a formulation of the present invention for use in preventing or inhibiting growth of EPEC in the digestive system of a subject in need thereof. The use may comprise administering to the subject the formulation according to the present invention.

The present invention is directed a formulation of the present invention for use in preventing or inhibiting bacterial infection in the intestinal lumen of a subject in need thereof. Such an infection may be prevented or inhibited by preventing or inhibiting the growth of bacteria. The use may comprise administering to the subject the formulation according to the present invention.

The present invention is directed a formulation of the present invention for use in increasing IL-18 secretion by intestinal cells in a subject in need thereof. The use may comprise administering to the subject the formulation according to the present invention.

The present invention is directed a formulation of the present invention for use in increasing intestinal immunity in a subject in need thereof. Such an increase in intestinal immunity may be due to an increase in the section of IL-18 by intestinal cells. The use may comprise administering to the subject the formulation according to the present invention.

In some embodiments, the efficacy of the formulation of the present invention in the methods and uses described herein may be determined by identifying an at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% improvement or change relative to a measurement in a control. In some embodiments, the efficacy of the formulation of the present invention may be determined by establishing a significant difference from a control, in which p<0.10, or p<0.05, or p<0.01. The control may be a normal healthy control, a pre-treatment control, or an earlier timepoint control.

### Administration of the Formulation

The formulation may be administered orally to a subject in need thereof. In embodiments in which the formulation is in a powder form, the formulation may first be reconstituted with an appropriate amount of water (according to the package directions) to a caloric density of 20-24 cal/oz, depending on the formula type. Alternatively, the formulation may be sprinkled onto foods or added to human breast milk if used as a fortifier.

In some embodiments, the formulation may be expelled directly into a subject's intestinal tract. For example, the formulation may be expelled directly into the gut. In some embodiments, the composition may be administered enterally under the supervision of a physician and may be intended for the specific dietary management of a disease or condition, such as celiac disease and/or food allergy.

In embodiments in which the formulation is an infant formula, the formulation may be delivered to an infant starting at birth. Delivery of the formulation may continue, in some embodiments, through no later than about two years of age; for example, through about one month, or about three months, or about six months, or about nine months, or about one year, or about 15 months, or about 18 months, or about 21 months, about 2 years, or iterations therebetween. In certain embodiments, the infant formula may be administered until the infant has transitioned fully to solid foods, although in some embodiments, the infant formula may continue to be administered as a supplemental source of nutrition.

In embodiments of the invention, the formulation may be administered to infants who were born prematurely. The formulation may be administered until a time that matches full-term gestation. Or, the formulation may be delivered to an infant until at least about three months corrected age, about six months corrected age, about nine months corrected age, about one year corrected age, about 15 months corrected age, about 18 months corrected age, about 21 months corrected age, about two years corrected age, or iterations therebetween. In some embodiments, the formulation may be delivered to a subject as long as is necessary to correct nutritional deficiencies.

In embodiments of the present invention, the formulation may used in a growing-up milk. Growing-up milks are fortified milk-based beverages intended for children over about 1 year of age (generally from about one to about three years of age, or from about four to about six years of age, or from about one year to about six years of age). Growing-up milks can compensate for nutritional deficiencies or can complement a diet to provide additional insurance that a child is receiving all necessary nutrition.

In some embodiments, the formulation may be administered as a daily or multiple-times-a-day supplement.

### Preparation of the Formulation

The formulations of the present invention may be prepared by mixing the components of the formulation together. In some embodiments, generally, essential nutrients and other components may be compounded in a wet process and then spray dried, after which certain heat-sensitive ingredients such as vitamins and lactoferrin may be dry-blended into the spray-dried powder. The dried powder product may be packaged and sealed and held until it undergoes a final check for conformance to specifications and regulations, including testing for microbiological contaminants. Liquid ready-to-feed and concentrated liquid infant formula may be processed as a low-acid food and must meet additional processing regulations as well.

The WPH protein may be provided from various sources, such as a commercially available Whey protein hydrolysate that would be Generally Recognized as Safe (GRAS) (the same would be true for all the various ingredients envisioned in this application). For example, in some embodiments, WPH protein may be provided in the formulation using Whey Protein Hydrolysate (DI3071). In certain embodiments WPH protein may be provided in the formulation using Whey Protein Hydrolysate 8350 which is derived from Hilmar Ingredients.

In certain embodiments, the alpha-lactalbumin is provided in the formulation using Lacprodan® ALPHA-10 or ALPHA-20 in the formulation. Both ALPHA-10 and ALPHA-20 are a native whey protein isolate that contains alpha-lactalbumin among other components. ALPHA-10 comprises 43% alpha-lactalbumin, and ALPHA-20 comprises 60% alpha-lactalbumin.

In certain embodiments, lactoferrin is provided in the formulation using Glanbia Nutritionals Inc.'s Bioferrin 1000® or Bioferrin 2000®. Bioferrin 2000® comprises 93% lactoferrin.

In certain embodiments, osteopontin is provided by including Lacprodan® OPN-10 which comprises 86% osteopontin.

Notably, alpha-lactalbumin, lactoferrin, and osteopontin are also provided in the formulation using whole milk, such as bovine whole milk.

An aspect of the present invention is directed to a method of preventing or reducing digestion of lactoferrin, osteopontin, or a combination thereof, including a lactoferrin-osteopontin complex, in a nutritional formulation. The method may comprise adding WPH protein, alpha-lactalbumin, or a combination thereof, to the nutritional formulation in an amount that results in the formulations of the present invention described herein, i.e., to achieve the weight ratios or g/L in the formulations of the present invention.

The present invention is also directed to a method of maintaining or increasing the amount of intact lactoferrin, osteopontin, or a combination thereof, including a lactoferrin-osteopontin complex, in a nutritional formulation. The method may comprise adding WPH protein, alpha-lactalbumin, or a combination thereof, to the nutritional formulation in an amount that results in the formulations of the present invention described herein, i.e., to achieve the weight ratios or g/L in the formulations of the present invention.

### EXAMPLES

### Example 1

This example illustrates different formulations having a protein component that comprises one or more of digestion-aiding WPH protein, digestion-aiding alpha-lactalbumin, immunoprotective lactoferrin, and immunoprotective osteopontin, according to embodiments of the invention. One of ordinary skill in the art would recognize that other digestion-aiding proteins and immunoprotective proteins as described throughout the specification may be substituted into these formulations.

Formulation 1 comprises a protein component that comprises WPH protein, alpha-lactalbumin, lactoferrin, and osteopontin, in which Lacprodan® ALPHA-10 is the source of the alpha-lactalbumin, in accordance to embodiments of the present invention. The nutrients in this formulation are shown in Table 1 below.

TABLE 1
Nutrients of Formulation 1.

| Nutrient | Quantity (per liter) | | Quantity (per 100 g) | |
|---|---|---|---|---|
| Protein | 13.4 | g | 10.0 | g |
| Alpha-lactalbumin | 2.4 | g | 1.8 | g |
| Lactoferrin | 0.85 | g | 0.64 | g |
| Osteopontin | 130.0 | mg | 97.5 | mg |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 1, the different protein sources and their percent contribution to the protein component is shown in Table 2 below.

TABLE 2
Protein sources and their percent contribution in Formulation 1.

| | % Ingredient |
|---|---|
| | Protein to Total |
| Ingredient | Formula Protein |
| Lacprodan ® DI-3071 (whey protein hydrolysate) | 35.0 |
| Lacprodan ® Alpha-10 whey protein concentrate | 41.8 |
| of which alpha-lactalbumin contributes | 17.9 |
| Bioferrin ® 2000 (lactoferrin) | 6.36 |
| Lacprodan ® OPN-10 (osteopontin) | 0.94 |
| Whole Milk | 15.9 |
| Total | 100.0 |
| Whey-casein ratio | 87.2:12.8 |

Formulation 2 comprises a protein component that comprises WPH protein, alpha-lactalbumin, lactoferrin, and osteopontin, in which Lacprodan® ALPHA-20 is the source of the alpha-lactalbumin, in accordance to embodiments of the present invention. The nutrients in this formulation are shown in Table 3 below.

TABLE 3
Nutrients of Formulation 2.

| Nutrient | Quantity (per liter) | | Quantity (per 100 g) | |
|---|---|---|---|---|
| Protein | 13.4 | g | 10.0 | g |
| Alpha-lactalbumin | 2.4 | g | 1.8 | g |
| Lactoferrin | 0.85 | g | 0.64 | g |
| Osteopontin | 130.0 | mg | 97.5 | mg |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 2, the different protein sources and their percent contribution to the protein component is shown in Table 4 below.

TABLE 4
Protein sources and their percent contribution in Formulation 2.

| | % Ingredient |
|---|---|
| | Protein to Total |
| Ingredient | Formula Protein |
| Lacprodan ® DI-3071 (whey protein hydrolysate) | 45.0 |
| Lacprodan ® Alpha-20 whey protein concentrate | 29.9 |
| of which alpha-lactalbumin contributes | 17.9 |
| Bioferrin ® 2000 (lactoferrin) | 6.36 |
| Lacprodan ® OPN-10 (osteopontin) | 0.94 |
| Whole Milk | 15.9 |
| Total | 100.0 |
| Whey-casein ratio | 85.8:14.2 |

Formulation 3 comprises a protein component that comprises WPH protein and alpha-lactalbumin, in which Lacprodan® ALPHA-10 is the source of the alpha-lactalbumin, in accordance to embodiments of the present invention. The nutrients in this formulation are shown in Table 5 below.

TABLE 5
Nutrients of Formulation 3.

| Nutrient | Quantity (per liter) | | Quantity (per 100 g) | |
|---|---|---|---|---|
| Protein | 13.4 | g | 10.0 | g |
| Alpha-lactalbumin | 2.4 | g | 1.8 | g |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 3, the different protein sources and their percent contribution to the protein component is shown in Table 6 below.

TABLE 6
Protein sources and their percent contribution in Formulation 3.

| | % Ingredient |
|---|---|
| | Protein to Total |
| Ingredient | Formula Protein |
| Lacprodan ® DI-3071 (whey protein hydrolysate) | 35.0 |
| Lacprodan ® Alpha-10 whey protein concentrate | 41.8 |
| of which alpha-lactalbumin contributes | 17.9 |
| Whole Milk | 23.2 |
| Total | 100.0 |
| Whey-casein ratio | 81.4:18.6 |

Formulation 4 comprises a protein component that comprises lactoferrin and osteopontin, in accordance to embodiments of the present invention. The nutrients in this formulation are shown in Table 7 below.

TABLE 7
Nutrients of Formulation 4.

| Nutrient | Quantity (per liter) | | Quantity (per 100 g) | |
|---|---|---|---|---|
| Protein | 13.4 | g | 10.0 | g |
| Lactoferrin | 0.85 | g | 0.64 | g |
| Osteopontin | 130.0 | mg | 97.5 | mg |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 4, the different protein sources and their percent contribution to the protein component is shown in Table 8 below.

TABLE 8
Protein sources and their percent contribution in Formulation 4.

| | % Ingredient |
|---|---|
| | Protein to Total |
| Ingredient | Formula Protein |
| Whey protein concentrate-35 | 70.0 |
| Bioferrin ® 2000 (lactoferrin) | 6.4 |
| Lacprodan ® OPN-10 (osteopontin) | 0.94 |
| Whole Milk | 22.7 |
| Total | 100.0 |
| Whey-casein ratio | 81.8:18.2 |

Formulation 5 comprises a protein component that comprises osteopontin, in accordance to embodiments of the present invention. The nutrients in this formulation are shown in Table 9 below.

TABLE 9
Nutrients of Formulation 5.

| Nutrient | Quantity (per liter) | | Quantity (per 100 g) | |
|---|---|---|---|---|
| Protein | 13.4 | g | 10.0 | g |
| Osteopontin | 130.0 | mg | 97.5 | mg |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 5, the different protein sources and their percent contribution to the protein component is shown in Table 10 below.

TABLE 10
Protein sources and their percent contribution in Formulation 5.

| | % Ingredient |
|---|---|
| | Protein to Total |
| Ingredient | Formula Protein |
| Whey protein concentrate-35 | 80.0 |
| Lacprodan ® OPN-10 (osteopontin) | 0.94 |
| Whole Milk | 19.1 |
| Total | 100.0 |
| Whey-casein ratio | 84.8:15.2 |

Formulation 6 comprises a protein component that comprises alpha-lactalbumin and osteopontin, in accordance to embodiments of the present invention. The nutrients in this formulation are shown in Table 11 below.

TABLE 11
Nutrients of Formulation 6.

| Nutrient | Quantity (per liter) | | Quantity (per 100 g) | |
|---|---|---|---|---|
| Protein | 13.4 | g | 10.0 | g |
| Alpha-lactalbumin | 2.4 | g | 1.8 | g |
| Osteopontin | 130.0 | mg | 97.5 | mg |
| Fat | 37.3 | g | 28.0 | g |
| Linoleic Acid | 7308.3 | mg | 5481 | mg |
| AA | 281 | mg | 211 | mg |
| DHA | 141 | mg | 105.5 | mg |
| Carbohydrate | 76.0 | g | 57.0 | g |
| GOS | 4.0 | g | 3.0 | g |
| Vitamins - Fat Soluble | | | | |
| Vitamin A | 2111 | IU | 1583 | IU |
| Vitamin D | 422 | IU | 317 | IU |
| Vitamin E | 14.1 | IU | 10.6 | IU |
| Vitamin K | 63.3 | μg | 47.5 | μg |
| Vitamins - Water Soluble | | | | |
| Thiamine (Vitamin B₁) | 563 | μg | 422 | μg |
| Riboflavin | 985 | μg | 739 | μg |
| Vitamin B₆ | 422 | μg | 317 | μg |
| Vitamin B₁₂ | 2.1 | μg | 1.6 | μg |
| Niacin | 7035 | μg | 5276 | μg |
| Folic Acid | 113 | μg | 84.4 | μg |
| Pantothenic Acid | 3518 | μg | 2638 | μg |
| Biotin | 21.1 | μg | 15.8 | μg |
| Vitamin C | 84.4 | mg | 63.3 | mg |
| Choline | 169 | mg | 127 | mg |
| Inositol | 42.2 | mg | 31.7 | mg |
| Minerals | | | | |
| Calcium | 562.8 | mg | 422.1 | mg |
| Phosphorus | 323.6 | mg | 242.7 | mg |
| Magnesium | 56.3 | mg | 42.2 | mg |
| Iron | 7.7 | mg | 5.8 | mg |
| Zinc | 6.3 | mg | 4.7 | mg |
| Manganese | 105.5 | μg | 79.1 | μg |
| Copper | 527.6 | μg | 395.7 | μg |
| Iodine | 105.5 | μg | 79.1 | μg |
| Selenium | 19.7 | μg | 14.8 | μg |
| Sodium | 253.3 | mg | 190.0 | mg |
| Potassium | 759.8 | mg | 569.9 | mg |
| Chloride | 443.2 | mg | 332.4 | mg |

For Formulation 6, the different protein sources and their percent contribution to the protein component is shown in Table 12 below.

TABLE 12
Protein sources and their percent contribution in Formulation 6.

| | % Ingredient |
|---|---|
| | Protein to Total |
| Ingredient | Formula Protein |
| Whey protein concentrate-35 | 38.0 |
| Lacprodan ® Alpha-10 whey protein concentrate | 41.8 |
| of which alpha-lactalbumin contributes | 17.9 |
| Lacprodan ® OPN-10 (osteopontin) | 0.94 |
| Whole Milk | 19.3 |
| Total | 100.0 |
| Whey-casein ratio | 84.6:15.4 |

Formulation 7 comprises a protein component that comprises whey protein hydrolysate, alpha-lactalbumin, lactoferrin, and osteopontin, in accordance to embodiments of the present invention. The different protein sources and their percent contribution to the protein component are shown in Table 13 below.

TABLE 13
Protein sources and their percent contribution in Formulation 7.

| | % Ingredient |
|---|---|
| | Protein to Total |
| Ingredient | Formula Protein |
| Lacprodan ® DI-3071 (whey protein hydrolysate) | 26.0 |
| Lacprodan ® Alpha-10 whey protein concentrate | 41.8 |
| of which alpha-lactalbumin contributes | 17.9 |
| Bioferrin ® 2000 (lactoferrin) | 6.36 |
| Lacprodan ® OPN-10 (osteopontin) | 0.94 |
| Whole Milk | 24.9 |
| Total | 100.0 |
| Whey-casein ratio | 80:20 |

### Example 2

This example illustrates a fat blend according to embodiments of the invention. The fat blend is presented in Table 14 below, and the fatty acid profile of the fat blend is presented in Table 15 below.

TABLE 14
Fat blend.

| | Rapeseed | | High Oleic | | | | | | Innate Fat | |
|---|---|---|---|---|---|---|---|---|---|---|
| | Oil (low | Sunflower | Sunflower | Coconut | | | Whole | | from Other | Calculated |
| | erucic acid) | Oil | Seed Oil | Oil | ARASCO | DHASCO-B | Milk | Monoglycerides | Sources | Total Fat |
| Total Fat (g/L) | 9.77 | 3.26 | 13.02 | 6.51 | 0.59 | 0.38 | 4.38 | 0.33 | 0.32 | 38.6 |
| % Total Fat | 25.3 | 8.4 | 33.8 | 16.9 | 1.54 | 0.99 | 11.4 | 0.9 | 0.8 | 100 |

TABLE 15
Fatty acid profile of the fat blend (values are % of the fatty acid in the fat blend).

| | | | Contribution from Oils in the Fat Blend | | | | | | |
|---|---|---|---|---|---|---|---|---|---|
| | | | Rapeseed | | High Oleic | | | | |
| | | | Oil (low | Sunflower | Sunflower | Coconut | | | Whole |
| | | Fat Blend | erucic acid) | Oil | Seed Oil | Oil | ARASCO | DHASCO-B | Milk |
| 6:0 Caproic | C6:0 | 0.17 | 0.00 | 0.00 | 0.00 | 0.00 | 0.00 | 0.00 | 0.17 |
| 8:0 Caprylic | C8:0 | 1.45 | 0.00 | 0.00 | 0.00 | 1.28 | 0.00 | 0.00 | 0.17 |
| 10:0 Capric | C10:0 | 1.39 | 0.00 | 0.00 | 0.00 | 1.00 | 0.00 | 0.00 | 0.39 |
| 12:0 Lauric | C12:0 | 8.70 | 0.00 | 0.00 | 0.00 | 8.23 | 0.00 | 0.00 | 0.47 |
| 14:0 Myristic | C14:0 | 4.84 | 0.00 | 0.01 | 0.00 | 3.36 | 0.02 | 0.00 | 1.46 |
| 16:0 Palmitic | C16:0 | 8.16 | 1.06 | 0.56 | 1.40 | 1.63 | 0.16 | 0.00 | 3.35 |
| 16:1 Palmitoleic | C16:1 | 0.28 | 0.05 | 0.01 | 0.03 | 0.00 | 0.02 | 0.00 | 0.17 |
| 18:0 Stearic | C18:0 | 3.77 | 0.50 | 0.27 | 1.13 | 0.47 | 0.16 | 0.00 | 1.23 |
| 18:1 Oleic | C18:1w9 | 48.14 | 16.08 | 2.93 | 27.65 | 1.15 | 0.32 | 0.00 | 0.00 |
| 18:2 Linoleic | C18:2w6 | 14.40 | 5.06 | 4.87 | 4.05 | 0.29 | 0.00 | 0.00 | 0.13 |
| 18:3 Linolenic | C18:3w3 | 2.83 | 2.66 | 0.00 | 0.04 | 0.01 | 0.04 | 0.00 | 0.08 |
| 20:0 Arachidic | C20:0 | 0.34 | 0.20 | 0.02 | 0.10 | 0.00 | 0.00 | 0.00 | 0.01 |
| 20:1 Eicosenoic | C20:1 | 0.41 | 0.28 | 0.03 | 0.10 | 0.00 | 0.00 | 0.00 | 0.00 |
| 20:2 Eicosadienoic | C20:2 | 0.00 | 0.00 | 0.00 | 0.00 | 0.00 | 0.00 | 0.00 | 0.00 |
| 20:3 Eicosatrienoic | C20:3w6 | 0.04 | 0.00 | 0.00 | 0.00 | 0.00 | 0.04 | 0.00 | 0.00 |
| 20:4 Arachidonic | C20:4w6 | 0.64 | 0.00 | 0.00 | 0.00 | 0.00 | 0.64 | 0.00 | 0.00 |
| 20:5 Eicosapentaenoic | | 0.00 | 0.00 | 0.00 | 0.00 | 0.00 | 0.00 | 0.00 | 0.00 |
| 22:0 Behenic | | 0.47 | 0.08 | 0.04 | 0.34 | 0.00 | 0.00 | 0.00 | 0.01 |
| 22:1 Erucic | | 0.00 | 0.00 | 0.00 | 0.00 | 0.00 | 0.00 | 0.00 | 0.00 |
| 22:5 Docosapentaenoic | | 0.00 | 0.00 | 0.00 | 0.00 | 0.00 | 0.00 | 0.00 | 0.00 |
| 22:6 Docosahexaenoic | C22:6w3 | 0.41 | 0.00 | 0.00 | 0.00 | 0.00 | 0.00 | 0.41 | 0.00 |
| 24:0 Lignoceric | C24:0 | 0.04 | 0.03 | 0.00 | 0.00 | 0.01 | 0.00 | 0.00 | 0.00 |
| Total | | 96.48 | | | | | | | |

### Example 3

The following study assessed the bioactivities of the bovine milk lactoferrin-osteopontin complex (at molecular ratios of lactoferrin:osteopontin of 3:1, 5:1, or 8:1) in formulas comprising mixtures of bovine lactoferrin (Bioferrin® 2000 , Glanbia Nutritionals, Inc., Fitchburg, Wis., osteopontin (Arla Foods Ingredients, Viby, Denmark), bovine whey protein hydrolysate, and α-lactalbumin (Lacprodan® ALPHA-10, Arla Foods Ingredients, Viby, Denmark).

### Formulations

The formulations prepared for this study are summarized in Table 14.

TABLE 14
Summary of the formulations studied in Example 3.

| | | |
|---|---|---|
| | Lactoferrin and/or | Additional |
| Formulation | Osteopontin | Proteins |

Single Component

| | |
|---|---|
| 1 | Lactoferrin only |
| 2 | Osteopontin only |

Lactoferrin + Osteopontin Mixtures

| | |
|---|---|
| 3 | Lactoferrin and osteopontin |
| | (3:1 molecular ratio) |
| 4 | Lactoferrin and osteopontin |
| | (5:1 molecular ratio) |
| 5 | Lactoferrin and osteopontin |
| | (8:1 molecular ratio) |

Protein Blends

| | | |
|---|---|---|
| 6 | Lactoferrin and osteopontin | Whey protein hydrolysate |
| | (3:1 molecular ratio) | and alpha-lactalbumin |
| 7 | Lactoferrin and osteopontin | Whey protein hydrolysate |
| | (5:1 molecular ratio) | and alpha-lactalbumin |
| 8 | Lactoferrin and osteopontin | Whey protein hydrolysate |
| | (8:1 molecular ratio) | and alpha-lactalbumin |

The composition of the protein blends are summarized in Table 15.

TABLE 15
Composition of the protein blends.

| Molecular | Amount (g/L) | | | |
|---|---|---|---|---|
| ratio of | | | Whey | |
| lactofer- | | | protein | Alpha- |
| rin:osteopontin | Lactoferrin | Osteopontin | hydrolysate | lactalbumin |
| 3:1 | 0.51 | 0.13 | 3.68 | 1.03 |
| 5:1 | 0.85 | 0.13 | 3.68 | 1.03 |
| 8:1 | 1.36 | 0.13 | 3.68 | 1.03 |

The lactoferrin, osteopontin, whey protein hydrolysate, and alpha-lactalbumin in each formulation were dissolved in Milli-Q water. For all experiments, lactoferrin, osteopontin, lactoferrin+osteopontin mixtures, and the protein blends were diluted proportionally, which means that the concentrations shown in Table 15 are 1×; when the samples are diluted 10× and 5×, the concentrations are 0.1× and 0.2×, respectively. To isolate the lactoferrin-osteopontin complex from the lactoferrin+osteopontin mixtures and the protein blends, the mixtures and blends were prepared by mixing bovine lactoferrin and osteopontin, and free lactoferrin and osteopontin were removed by using a Centriplus® centrifugal filter device (100,000 MWCO; YM-100, Millipore Corporation, Badford, Mass.) according to the manufacturer's instructions. The top fraction was collected and washed with ice-cold 1×PBS three times. The complex was identified by Blue native PAGE and Western blotting.

### Statistical Analysis

Results represent mean ±standard deviation from two to three independent experiments. Statistical analysis was performed using GraphPad Prism version 7.0 (GraphPad Software, San Diego, Calif.). One-way ANOVA was used to determine significant differences among groups. P<0.05 was considered as significant.

### Blue Native PAGE and Western Blotting—Methods and Results

The complex was identified by native gel electrophoresis using the NativePAGE™ Novex® Bis-Tris Gel System (Thermo Fisher Scientific, Rockford, Ill.), and 4% to 16% native Bis-Tris gels (Thermo Fisher Scientific) were used following the manufacturer's instructions for Coomassie Staining of the Native PAGE gels. 0.2× of each protein sample per lane was loaded.

Western blotting was performed to verify the lactoferrin-osteopontin complex and to examine whether the lactoferrin-osteopontin complex is resistant to in vitro digestion. Protein concentrations were measured by a Pierce™ BCA Protein Assay Kit (Thermo Fisher Scientific). Protein samples were mixed with 1×Laemmli sample buffer containing (5% β-mercaptoethanol) and boiled for five minutes. The Mini-PROTEAN® TGX Stain-Free gel (Bio-Rad, Hercules, Calif.) was used for SDS-PAGE, and the proteins were transferred to nitrocellulose membranes by the Turbo Blotting system (Bio-Rad). Membranes were blocked using a blocking solution (3% bovine serum albumin in 1×TBST; 1×Tris buffered saline with 1% Tween-20), and incubated for one hour at room temperature with an HRP conjugated antibody to lactoferrin (1:5000, A10-126P, Bethyl Laboratories, Montgomery, Tex.) or a primary antibody to osteopontin (anti-mouse, 1:2000, MAB193P, Maine Biotechnology, Portland, Me.) in blocking buffer. After three washes with 1×TBST, membranes were incubated for one hour at room temperature with or without a horseradish peroxidase-conjugated anti-mouse IgG secondary antibody, at a dilution of 1:20,000. The ECL Plus reagent (Thermo Scientific, Waltham, Mass.) was used to visualize the proteins.

As shown in FIGS. 1-3, the complex appears in the three mixtures and the three protein blends. Based on FIGS. 2 and 3, free lactoferrin and osteopontin exist in both the mixtures and the protein blends, and multiple complex bands are observed ≥720 kDa. Since the osteopontin concentration is fixed and 0.2×protein solutions were used for the blue native gels and immunoblotting, a similar density of the osteopontin band was expected to be seen in osteopontin only, the three lactoferrin+osteopontin mixtures, and the protein blends. Surprisingly, the osteopontin bands in the three protein blends are similar to the free osteopontin sample and is more intense than the osteopontin band in the three lactoferrin+osteopontin mixtures, suggesting that the formula protein base (whey protein hydrolysate and alpha-lactalbumin) helps keep the osteopontin intact during sample processing for blue native PAGE and immunoblotting including running a PAGE gel under a native condition for two hours and transfer of all proteins from the native gel to a PVDF membrane. Lactoferrin and osteopontin have the same molecular weights in the complex as the individual proteins alone. Less osteopontin was detected in the complex when the ratio of lactoferrin to osteopontin increased. Lactoferrin in the complex showed bands of 80, 64, 50, and 35 kDa by SDS-PAGE, of which the 80 kDa band dominated (FIG. 4). Further, more lactoferrin was detected in the complex when the ratio of lactoferrin to osteopontin increased. As shown in FIG. 5, osteopontin mostly showed a band of 40 kDa, and a smaller band of 63 kDa.

### In Vitro Digestion—Methods and Results

Lactoferrin, osteopontin, lactoferrin+osteopontin mixtures, and the three protein blends (1 mg/mL and 1 mL of each) were dissolved in Milli-Q water (Millipore, Bedford, Mass.), respectively. The samples were adjusted to pH 4.0 with 1 M HCl and were mixed with 2% porcine pepsin in 1 mM HCl (Sigma) at a pepsin/protein ratio of 1:12.5. After shaking at 120 rpm for 30 minutes at 37° C. in the dark to mimic gastric incubation, samples were immediately adjusted to pH 7.0 with 1M NaHCO₃, followed by addition of 0.4% porcine pancreatin in 0.1M NaHCO₃ (Sigma) at a pancreatin/protein ratio of 1:62.5. Samples were shaken at 120 rpm for 30 minutes at 37° C. in the dark and then immediately subjected to an 85° C. water bath for three minutes and mixed with 2×Laemmli sample buffer (Bio-Rad) containing β-mercaptoethanol (5%) and boiled for 5 minutes. The boiled samples were then subjected to Western blotting. 20 μL of the digested samples were used to detect survived lactoferrin and then concentrated by centrifugation using YM-3 (3K MWCO; Millipore, Bedford, Mass.) at 3000 g for 30 min for detection of osteopontin (20 μL of each). Non-digested lactoferrin (1 μg/lane) or osteopontin (0.3 μg/lane) were loaded as controls.

As shown in FIGS. 6 and 7, lactoferrin and osteopontin were digested into smaller fragments. After digestion, there was still intact lactoferrin, and as the ratio of lactoferrin and osteopontin increased, more intact lactoferrin survived. Further, lactoferrin was digested into fragments of 52, 45, 35, and 32 kDa in all groups (FIG. 6). The more lactoferrin in the complex, the more lactoferrin survived, and lactoferrin resisted in vitro digestion the most in the group with a ratio of 8:1. FIG. 7 shows the digestibility of osteopontin, which was digested into small fragments.

The amount of lactoferrin was similar in the lactoferrin standard and the 3:1 sample before digestion, but more lactoferrin survived in the 3:1 sample than in the lactoferrin-only sample after digestion, suggesting that peptides and proteins in whey protein hydrolysate and alpha-lactalbumin protect lactoferrin from in vitro digestion.

### HIEC Cell Proliferation—Methods and Results

Human intestinal crypt-like cells (HIECs) were cultured in Opti-MEM (Life Technologies Inc., Gaithersburg, Md.) with 5% fetal bovine serum (FBS), 2.4 g/L NaHCO₃ (Sigma), 0.5% penicillin/streptomycin solution (Gemini Bio-products, West Sacramento, Calif.), 1% HEPES buffer (Thermo Fisher Scientific), 1% GlutaMax (Thermo Fisher Scientific), and 0.05 EGF (Thermo Fisher Scientific) in a humidified cell culture incubator with 5% CO₂ at 37° C.

HIECs (˜40% confluence) were fasted for 2 hours with serum free medium (SFM) and then cell culture medium containing 1% FBS treated with lactoferrin, osteopontin, lactoferrin+osteopontin mixtures, and the protein blends in cell culture medium containing 1% FBS. Cellular proliferation was measured using a bromodeoxyuridine (BrdU) ELISA kit (Roche, Indianapolis, Ind.) according to the manufacturer's instructions.

The protein blends with different ratios of lactoferrin to osteopontin exhibited different effects on HIEC proliferation (FIG. 8). When the ratio of lactoferrin to osteopontin was 3:1, the blends significantly increased HIEC proliferation and showed stronger promotion than the 5:1 group at all concentrations and than the 8:1 group at two concentrations (13:0.1× and 6.5:0.05×).

### Caco-2 Cell Differentiation—Methods and Results

Human colon adenocarcinoma cells (Caco-2, American Type Collection, Rockville, Md.) were used to assess effects of the protein blends on differentiation of intestinal cells. These cells were cultured in MEM (GE Healthcare) with 10% FBS, 2.2 g/L NaHCO₃, 1% penicillin/streptomycin solution (Gemini) in a humidified cell culture incubator with 5% CO₂ at 37° C. Experiments were conducted on cells between passages 25 to 30 and the cell culture medium was changed every 2 days.

Caco-2 cells (˜90% confluence) were fasted with SFM for two hours and then incubated with 0.05× of lactoferrin, osteopontin, lactoferrin+osteopontin mixtures, and protein blends at different concentrations (0.00625×, 0.0125×, 0.025×, and 0.05×) of the protein blends in cell culture medium with 1% FBS at 37° C. for 72 hours. After cells were washed three times with ice-cold 1×PBS, cells were homogenized in lysis buffer (1×PBS containing 0.1% Triton X-100, and 1×Roche complete protease inhibitor cocktail). Alkaline phosphatase activity was measured using pNPP as a substrate (Thermo Fisher).

Differentiation of Caco-2 cells was significantly promoted except for the 8:1 protein blend-treated group. The lower concentrations of lactoferrin (3:1 and 5:1) displayed greater effects on cellular differentiation than 8:1 lactoferrin, which suggests that a lower concentration of lactoferrin is required for promotion of cellular differentiation. Consistent with the lactoferrin results, the lactoferrin+osteopontin mixture and the protein blend with a 3:1 ratio exhibited the strongest enhancing effects on cellular differentiation compared with the 5:1 and 8:1 ratios. The 8:1 protein blend did not enhance differentiation of Caco-2 cells likely due to the high concentration of lactoferrin and an interaction of lactoferrin and osteopontin with ingredients from whey protein hydrolysate and alpha-lactalbumin.

### Lactoferrin Internalization—Methods and Results

Differentiated Caco-2 cells (day 13) were used to assess lactoferrin internalization (binding and uptake) of the lactoferrin-only formulation, the lactoferrin+osteopontin mixtures, and the protein blends. The cells were treated with each of these formulations (0.1×) for 2 hours at 4° C. (binding) or at 37° C. (uptake). The cells were collected in PBS buffer with 0.01% Triton-x 100. The lactoferrin binding to cells or uptake by cells were measured using an enzyme-linked immunosorbent assay (ELISA).

The results presented in FIG. 10 (binding) and FIG. 11 (uptake) shows that both binding and uptake were greater as the ratio between lactoferrin and osteopontin increased. In addition, binding and uptake were greater by treatment with the lactoferrin+osteopontin mixtures as compared to the protein blends.

### EPEC Growth—Methods and Results

The effects of lactoferrin, osteopontin, lactoferrin+osteopontin mixtures, and protein blends on growth of EPEC (ATCC® BAA-2440™) were determined by counting Colony Forming Units (CFUs) on agar plates. To do so, samples of EPEC (5×10⁵ CFUs/mL) cultured with lactoferrin, osteopontin, lactoferrin+osteopontin mixtures, or protein blends (0.2× in Difco Nutrient Broth medium) were incubated for 16 hours at 37° C. with shaking at 160 rpm. Aliquots of the medium (10 μL) were plated onto Luria-Bertani (LB) agar, and CFUs were counted after overnight incubation at 37° C. OD600 was measured to evaluate growth of EPEC.

Based on the OD 600 results, peak inhibition occurred at 16 hours when the protein concentration was 0.2×. Therefore, 0.2× of lactoferrin, osteopontin, lactoferrin+osteopontin mixtures, and the three protein blends were incubated with EPEC for 16 h and colony forming units (CFUs) on LB agar were counted. As shown in FIGS. 12A-12C, growth of EPEC was inhibited by all these proteins. Lactoferrin-only formulation at the 5:1 ratio quantity, lactoferrin-only formulation at the 8:1 ratio quantity, and the lactoferrin+osteopontin mixture at the 8:1 ratio exhibited the most potent effect. Presence of whey protein hydrolysate and alpha-lactalbumin did not modify the anti-EPEC activity of the lactoferrin-osteopontin complex. The three lactoferrin+osteopontin mixtures variously attenuated growth of EPEC, and the lactoferrin+osteopontin mixture with an 8:1 ratio displayed the greatest inhibition. However, three protein blends showed similar effects on growth of EPEC, which may result from interactions with proteins and peptides from whey protein hydrolysate and alpha-lactalbumin.

### IL-18 Secretion—Methods and Results

Caco-2 cells (day 15) were fasted for 2 hours with SFM and then treated with 0.05× of lactoferrin, osteopontin, lactoferrin+osteopontin mixtures, or the protein blends in cell culture medium containing 1% FBS at 37° C. for 72 hours. Cell culture media were collected and concentrated by YM-3 (3K MWCO; Millipore) to measure concentrations of secreted IL-18. IL-18 was quantified by using an ELISA kit (R&D Systems, Minneapolis, Minn.) according to the manufacturer's instructions.

Secretion of IL-18 by Caco-2 cells was measured by an ELISA kit to assess involvement of the lactoferrin-osteopontin complex in intestinal immunity. As shown in FIG. 13, all the treatments promoted IL-18 secretion by Caco-2 cells. The IL-18 secretion in the 5:1 lactoferrin+osteopontin mixture treated group was significantly lower than the 3:1 and 8:1 lactoferrin+osteopontin mixture treated groups, and the three blends showed a trend similar to the lactoferrin+osteopontin mixtures.

The foregoing description is given for clearness of understanding only, and no unnecessary limitations should be understood therefrom, as modifications within the scope of the invention may be apparent to those having ordinary skill in the art.

Throughout this specification and the claims that follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A formulation for oral administration comprising a protein component, wherein the protein component comprises:
(a) lactoferrin, and
(b) osteopontin;
wherein the ratio by weight of the lactoferrin to the osteopontin is about 12:1 to about 2:1.

2. The formulation of claim 1, wherein the ratio by weight of the lactoferrin to the osteopontin is about 10:1 to about 3:1.

3. The formulation of claim 1, wherein the formulation further comprises one or more digestion-aiding proteins.

4. The formulation of claim 3, wherein the ratio by weight of the one or more digestion aiding proteins to the lactoferrin and osteopontin is about 12:1 to about 1:1.

5. (canceled)

6. The formulation of claim 3, wherein the one or more digestion-aiding proteins comprise whey protein hydrolysate, alpha-lactalbumin, or a combination thereof.

7. A formulation for oral administration comprising a protein component, wherein the protein component comprises:
(a) one or more digestion aiding proteins comprising whey protein hydrolysate, alpha-lactalbumin, or a combination thereof; and
(b) one or more immunoprotective proteins comprising lactoferrin, osteopontin, or a combination thereof;
wherein the ratio by weight of the one or more digestion aiding proteins to the one or more immunoprotective proteins is about 12:1 to about 1:1.

8. The formulation of claim 7, wherein the ratio by weight of the one or more digestion aiding proteins to the one or more immunoprotective proteins is about 10:1 to about 3:1.

9. The formulation of claim 7, wherein the one or more digestion aiding proteins comprises whey protein hydrolysate, and the one or more immunoprotective proteins comprises lactoferrin; and
wherein the ratio by weight of the whey protein hydrolysate to the lactoferrin is about 9:1 to about 1:1.

10-11. (canceled)

12. The formulation of claim 7, wherein the one or more digestion aiding proteins comprises whey protein hydrolysate, and the one or more immunoprotective proteins comprises osteopontin; and
wherein the ratio by weight of the whey protein hydrolysate to the osteopontin is about 60:1 to about 10:1.

13-14. (canceled)

15. The formulation of claim 7, wherein the one or more digestion aiding proteins comprises alpha-lactalbumin, and the one or more immunoprotective proteins comprises lactoferrin; and
wherein the ratio by weight of the alpha-lactalbumin to the lactoferrin is about 4:1 to about 0.5:1.

16-17. (canceled)

18. The formulation of claim 7, wherein the one or more digestion aiding proteins comprises alpha-lactalbumin, and the one or more immunoprotective proteins comprises osteopontin; and
wherein the ratio by weight of the alpha-lactalbumin to the osteopontin is about 25:1 to about 5:1.

19-21. (canceled)

22. A method of providing nutritional support to a subject in need thereof, the method comprising administering to the subject a formulation according to claim 1.

23. A method of promoting proliferation of intestinal cells in a subject in need thereof, the method comprising administering to the subject a formulation according to claim 1.

24. A method of inducing proliferation of intestinal cells in a subject in need thereof, the method comprising administering to the subject a formulation according to claim 1.

25. A method of promoting differentiation of intestinal cells in a subject in need thereof, the method comprising administering to the subject a formulation according to claim 1.

26. A method of inducing differentiation of intestinal cells in a subject in need thereof, the method comprising administering to the subject a formulation according to claim 1.

27. (canceled)

28. A method of inhibiting growth of enteropathogenic Escherichia coli in the digestive system of a subject in need thereof, the method comprising administering to the subject a formulation according to claim 1.

29. (canceled)

30. A method of inhibiting bacterial infection in the intestinal lumen of a subject in need thereof, the method comprising administering to the subject a formulation according to claim 1.

31. A method of inhibiting growth of increasing interleukin-18 secretion by intestinal cells in a subject in need thereof, the method comprising administering to the subject a formulation according to claim 1.

32. A method of increasing intestinal immunity in a subject in need thereof, the method comprising administering to the subject a formulation according to claim 1.

33. A formulation for oral administration comprising a protein component, wherein the protein component comprises:
(a) one or more digestion aiding proteins comprising whey protein hydrolysate, alpha-lactalbumin, or a combination thereof; and
(b) lactoferrin;
wherein the ratio by weight of the one or more digestion aiding proteins to the lactoferrin is about 10:1 to about 0.5:1.

34. The formulation of claim 33, wherein the one or more digestion aiding proteins comprises whey protein hydrolysate; and
wherein the ratio by weight of the whey protein hydrolysate to the lactoferrin is about 9:1 to about 1:1.

35. The formulation of claim 33 wherein the one or more digestion aiding proteins comprises alpha-lactalbumin; and
wherein the ratio by weight of the alpha-lactalbumin to the lactoferrin is about 4:1 to about 0.5:1.
